(19) United States
(12) Patent Application Publication
(10) Pub. No.: US 2023/0210790 A1
(43) Pub. Date: Jul. 6, 2023

(54) GEL FOR USE IN GASTROINTESTINAL ENDOSCOPY AND ENDODERMAL, EPIDERMAL, AND OTHER MUCOSAL USES

(71) Applicants: The Johns Hopkins University, Baltimore, MD (US); University of Maryland, Baltimore, Baltimore, MD (US)

(72) Inventors: Venkata S. Akshintala, Baltimore, MD (US); Stephen W. Hoag, Catonsville, MD (US); Anthony N. Kalloo, Baltimore, MD (US); Ayesha Kamal, Baltimore, MD (US); Vikesh K. Singh, Ellicott City, MD (US); Mouen A. Khashab, Towson, MD (US)

(21) Appl. No.: 18/000,471

(22) PCT Filed: Jun. 2, 2021
(86) PCT No.: PCT/US21/35548

§ 371 Date: Dec. 1, 2022

Related U.S. Application Data
(60) Provisional application No. 63/033,441, filed on Jun. 2, 2020.

(51) Int. Cl. A61K 31/137; A61K 9/06; A61K 9/00; A61K 47/34; A61K 47/32; A61K 47/36; A61K 31/167

(52) U.S. Cl. CPC A61K 31/137; A61K 9/06; A61K 9/0019; A61K 47/34; A61K 47/32; A61K 47/36; A61K 31/167; A61B 2017/00269

(57) ABSTRACT

Disclosed are formulations comprising epinephrine, a degradable polymer, and, in some embodiments, a mucoadhesive compound, and methods of their use for preventing or controlling gastrointestinal bleeding and for facilitating endoscopic interventions for separating diseased tissue from normal tissue. Also disclosed in an endoscopic needle for administering the formulations.

### BACKGROUND

Gastrointestinal (GI) bleeding results in more than 400,000 annual hospitalizations in the United States alone. Gralnek et al, 2008. GI bleeding can result from pathologies, such as ulcers, cancerous lesions, mucosal tears, and the like. Endoscopy is effective in diagnosing and effectively managing most sources of GI bleeding including. Currently available endoscopic treatment modalities for GI bleeding include injection methods, for example, injection of hemostatic agents, cautery, and mechanical therapy, such as hemostat clip placement. Eisen et al., 2002. Among the injection therapies, the most widely-used agent, i.e., diluted epinephrine, is known to control bleeding through the tamponade effect, vasoconstriction of local vasculature, platelet aggregation, and stimulation of coagulation cascade.

Epinephrine's efficacy, however, is limited by its short duration of action. Extensive research using epinephrine with GI endoscopy exposed the significant shortcomings associated with the short action of epinephrine. Park et al., 2004; Sarmento et al., 2009. As a result of the shortcomings of epinephrine treatment alone, the American Society for Gastrointestinal Endoscopy (ASGE) has recommended the use of a second endoscopic therapy in conjunction with epinephrine, which adds to the cost of treatment. Hwang et al., 2012.

GI bleeding also can result from endoscopic interventions, such as colon polyp removal, sphincterotomy, and novel endoscopic interventions, which are increasingly replacing laparoscopic surgery for the treatment of, for example, achalasia (endoscopic myotomy), reflux disease (endoscopic fundoplication), and the like. Endoscopic interventions can involve separation of GI wall layers, tunneling, or third-space endoscopy, each of which can result in immediate or delayed bleeding. A clean operating field, a requirement which causes an increase in the duration of the procedure, is necessary during these endoscopic interventions for adequate visualization and to avoid complications.

### SUMMARY

The presently disclosed subject matter provides a sustained-release epinephrine formulation to effectively control GI bleeding as a stand-alone therapy.

In some aspects, the presently disclosed subject matter provides an injectable gel formulation for the sustained release of epinephrine, the formulation comprising epinephrine, or a pharmaceutically acceptable salt thereof, and a degradable polymer or a combination of polymers.

In some aspects, the degradable polymer or combination of polymers is selected from the group consisting of poly(lactic acid) (PLA), poly(DL-lactide) (PDLA), poly(dl-lactic acid), poly(DL-lactide-co-glycolide) (PLGA), poly(lactic-co-glycolic acid) (PLGA), poly(caprolactone) (PCL), poly(E-caprolactone), poly (ethylene oxide) (PEO), poly(P-dioxanone), poly(hydroxybutyrate), poly(B-malic acid), a poloxamer, poloxamer 407, polycarbophil and Ca++ salt(or equivalent salt), poly(methyl vinyl ether/maleic anhydride), a polyanhydride, a polyphosphazene, a poly(ortho ester), a poly(phosphoester), a polyhydroxyalkanoate (PHA), a polyurethane (PUR), a carbomer, cyclomethicone, chitosan, a triblock PEO-PPO-PEO copolymer of poly(ethylene oxide) (PEO) and poly(propylene oxide) (PPO), oxyethylene oxypropylene polymer (methyl oxirane polymer with oxirane), polyvinylpyrrolidone, alginic acid, Ca alginate and Na salt, agar, xanthan gum, chitosan, chitin, guar gum, a carrageenan, gellan gum, pregelatinized starch, a silk protein polymer, an elastine protein polymer, a silk-elastin protein polymer, collagen, hyaluronic acid, a pseudo-amino acid, albumin, fibrinogen, maltodextrin, tri-block copolymer/poloxamer, and gelatin.

In certain aspects, the injectable gel formulation comprises a polymer selected from the group consisting of chitosan, a triblock PEO-PPO-PEO copolymer of poly(ethylene oxide) (PEO) and poly(propylene oxide) (PPO), poly(D,L-lactide) (PDLA), poly(D,L-lactide-co-glycolide) (PLGA), oxyethylene oxypropylene polymer (methyl oxirane polymer with oxirane), oxyethylene oxypropylene polymer, poloxamer 407, and polyvinylpyrrolidone.

In particular aspects, the degradable polymer is methyl oxirane polymer with oxirane or poloxamer 407 alone or in combination with PEO.

In more particular aspects, the degradable polymer comprises from about 10% w/v to about 17% w/v PEO-PPO-PEO triblock copolymer. In other aspects, the degradable polymer comprises from about 0.8% w/v chitosan to about 5% w/v chitosan. In yet other aspects, the degradable polymer comprises about 2% w/v xanthan gum. In certain aspects, the degradable polymer comprises from about 13% w/v to about 17% w/v methyl oxirane polymer with oxirane. In other aspects, the degradable polymer comprises about 10% w/v polyvinylpyrrolidone.

In particular aspects, the degradable polymer comprises a mixture of about 17% w/v methyl oxirane polymer with oxirane and about 5% w/v chitosan (L) such that the final solution is in a ratio of 17:3 (17 mL of methyl oxirane polymer solution per 3 mL of chitosan(L) solution). In yet more particular aspects, the degradable polymer is selected from the group consisting of 1% w/v triblock PEO-PPO-PEO copolymer, 2% w/v triblock PEO-PPO-PEO copolymer, 5% w/v triblock PEO-PPO-PEO copolymer, 10% w/v triblock PEO-PPO-PEO copolymer, 11% w/v triblock PEO-PPO-PEO copolymer, 12% w/v triblock PEO-PPO-PEO copolymer, 13% w/v triblock PEO-PPO-PEO copolymer, 15% w/v triblock PEO-PPO-PEO copolymer, 0.8% w/v chitosan (L), 1% w/v chitosan (L), 1% w/v oxyethylene oxypropylene polymer, 10% w/v oxyethylene oxypropylene polymer, 1% w/v methyl oxirane polymer with oxirane, 10% w/v methyl oxirane polymer with oxirane, 13% w/v methyl oxirane polymer with oxirane, 15% w/v methyl oxirane polymer with oxirane, 1% w/v oxyethylene oxypropylene polymer, 10% w/v oxyethylene oxypropylene polymer, 1% w/v polyvinylpyrrolidone (L), 1% w/v polyvinylpyrrolidone (H), 10% w/v polyvinylpyrrolidone (H), and 15% w/v polyvinylpyrrolidone (H).

In yet more particular aspects, the injectable gel formulation comprises 6.5% w/v 13% triblock PEO-PPO-PEO copolymers of poly(ethylene oxide) (PEO) and poly(propylene oxide) (PPO), 1.0% w/v poly(ethylene oxide) (PEO), at least one buffer, 0.9% w/v NaCl, at least one dye (0.4 mg), ascorbic acid, and water.

In certain aspects, the injectable gel formulation further comprises from about 0.001 mg/mL to about 0.1 mg/mL epinephrine, either immediate release or an epinephrine-containing nanoparticle or a combination of both immediate release and an epinephrine-containing nanoparticle.

In other aspects, the injectable gel comprises a degradable polymer without epinephrine. In yet other aspects, the epinephrine comprises an epinephrine-containing nanoparticle.

In other aspects, the presently disclosed subject matter provides a mucoadhesive gel formulation for the sustained release of epinephrine, the formulation comprising epinephrine, a degradable polymer, and a mucoadhesive coating.

In some aspects, the degradable polymer is selected from the group consisting of poly(lactic acid) (PLA), poly(DL-lactide), poly(dl-lactic acid), poly(DL-lactide-co-glycolide), poly(lactic-co-glycolic acid) (PLGA), poly(caprolactone) (PCL), poly(E-caprolactone), poly(P-dioxanone), poly(hydroxybutyrate), poly(B-malic acid), poloxamer, polycarbophil and Ca++ salt, poly(methyl vinyl ether/maleic anhydride), a polyanhydride, a polyphosphazene, a poly(ortho ester), a poly(phosphoester), a polyhydroxyalkanoate (PHA), a polyurethane (PUR), a carbomer, cyclomethicone, alginic acid, Ca alginate and Na salt, agar, xanthan gum, chitosan, chitin, guar gum, a carrageenan, gellan gum, pregelatnaized starch, a silk protein polymer, an elastine protein polymer, a silk-elastin protein polymer, collagen, hyaluronic acid, a pseudo-amino acid, albumin, fibrinogen, maltodextrin, gelatin, polyethylene glycol in combination with acrylated poly-L lactid acid, trilyine amine, albumin, polyethyl amine, glutaraldehyde, polyaldehyde, cyanoacrylate, polyurethane, cyanoacrylate, dextran-urethanemethacrylate, sodium alginate conjugated either with 2-aminoethyl methacrylate, (AEMA), styryl-pyridine, methacrylic anhydride, acrylated poly(glycerol sebacate) (PGS), poly(vinyl acetate) (PVA), PEG, poly(c caprolactone) (PCL), acryloyl chloride (poly(glycerol sebacate acrylate) PGSA), and PEG diacrylate (PEG-DA).

In particular aspects, the degradable polymer is poly(lactic-co-glycolic acid (PLGA).

In certain aspects, the mucoadhesive coating is selected from the group consisting of chitosan, one or more chitosan salts, and one or more chitosan derivatives. In particular aspects, the mucoadhesive coating comprises chitosan.

In some aspects, the mucoadhesive gel formulation further comprises one or more hydrophobic components selected from the group consisting of a synthetic hydrophobic polymer, a naturally-occurring hydrophobic polymer, and combinations thereof. In certain aspects, the synthetic hydrophobic polymer is selected from the group consisting of a polyester, a polyurethane, a polyurea, a polycarbonate, a polyether, a polysulfide, a polysulfonate, a polyimide, a polybenzimidazole, and combinations thereof. In certain aspects, the naturally-occurring hydrophobic polymer is selected from a lipoglycan and a proteoglycan. In certain aspects, the synthetic hydrophobic polymer is selected from the group consisting of a polylactide, polyglycolide, poly(lactide-co-glycolide, poly(e-caprolactone), poly hydroxybutyrate, poly(dioxanone), poly(3-hydroxybutyrate), poly(3-hydroxyval crate), poly(valcrolactone), poly(tartonic acid), poly(malonic acid), poly(anhydrides), poly(orthoesters), polyphosphazenes and acryloyloxy dimethyl-y-butyrolactone (DBA) and other lactone-containing polymers, and combinations thereof. In certain aspects, the hydrophilic polymer is selected from the group consisting of a polyacrylic acid, a polyalcohol, a polyacrylate, a polyurethane, a polyacrylamine, a polyacrylamide, a polyether, and a polypyrollidone. In more certain aspects, the hydrophilic polymer comprises one or more monomers selected from the group consisting of acrylate, acrylic acid, methacrylate, methacrylic acid, methyl acrylate, ethyl acrylate, methyl methacrylate, acrylonitrile, 2-chloroethyl vinyl ether, 2-ethylhexyl acrylate, hydroxyethyl methacrylate, butyl acrylate, butyl methacrylate, trimethylolpropane triacrylate, hydroxypropylmethacrylamide, hydroxyethyl acrylate, poly(ethylene glycol) methacrylate, poly(N-isopropylacrylamide) (RNGRAM), poly(vinyl alcohol) (PVA), poly(-oxazoline), polyethylene glycol, polyvinylpyrollidone polymers, and copolymers thereof.

In some aspects, the mucoadhesive gel formulation further comprises one or more boronic acids selected from the group consisting of phenylboronic acid, 2-thienylboronic acid, methylboronic acid, cis-propenylboronic acid, trans-propenylboronic acid, (4-allylaminocarbonyl)benzeneboronic acid, (4-aminosulfonylphenyl)boronic acid, (4-benzyloxy-2-formyl)phenylboronic acid, (4-hydroxy-2-methyl)phenylboronic acid, (4-hydroxy-2-methyl)phenylboronic acid, (4-methanesulfonylaminomethylphenyl)boronic acid, (4-ethanesulfonylaminomethylphenyl)boronic acid, (4-methylaminosulfonylphenyl) boronic acid, (4-methylaminosulfonylphenyl)boronic acid, (4-phenylaminocarbonylphenyl) boronic acid, (4-henylaminocarbonylphenyl)boronic acid, (4-sec-butyl) benzeneboronic acid, (2,6-dimethoxy-4-methylphenyl)boronic acid, (2,6-dimethoxy-4-methylphenyl)boronic acid, (2-methylpropyl)boronic acid, (2-methylpropyl) boronic acid,(3-acetamido-5-carboxy)phenylboronic acid, (3-acetamido-5-carboxy) phenyl boronic acid, (3-acetamidomethylphenyl)boronic acid, (3-acetamidomethylphenyl) boronic acid, (3-allylaminocarbonyl)benzeneboronic acid, (3-cyanomethylphenyl)boronic acid, and derivatives thereof.

In certain aspects, the derivative of the one or more boronic esters is selected from the group consisting of allylboronic acid pinacol ester, phenyl boronic acid trimethylene glycol ester, diisopropoxymethylborane, bis(hexyleneglycolato)diboron, t-butyl-N-[4-(4,4,5,5-tetramethyl-1,3,2-dioxaborolan-2-yl)phenyl]carbamate, 2,6-dimethyl-4-(4,4,5,5-tetramethyl-1,3,2-dioxaborolan-2-yl)benzoate, 4-(4,4,5,5-tetramethyl-1,3,2-dioxaborolan-2-yl)aniline, 4-(4,4,5,5-tetramethyl-1,3,2-dioxaborolan-2-yl)benzoic acid, 4-(4,4,5,5-tetramethyl-1,3,2-dioxaborolan yl)phenol, 2-methoxy-4-(4,4,5,5-tetramethyl-1,3,2-dioxaborolan-2-yl)phenol, and combinations thereof.

In other aspects, the degradable polymer can be activated by chemical or UV-A light. In representative aspects, the degradable polymer comprises a mixture of PEO and 3,4-dihydroxyphenyl-L-alanine (DOPA). In certain aspects, the PEO-DOPA mixture further comprises sodium alginate. In more certain aspects, the PEO-DOPA mixture comprising sodium alginate has been activated with UV-A light and a photoinitiator. In particular aspects, the photoinitiator is 2-hydroxy-4′-(2-hydroxyethoxy)-2-methylpropiophenone or 2-hydroxy-1-(4-(2-hydroxyethoxy)phenyl)-2-methylpropan-1-one.

In other aspects, the degradable polymer is crosslinked by activation using a divalent or trivalent cation. In certain aspects, the divalent cation is selected from the group consisting of Ca²⁺, Mg²⁺, Ba²⁺, Sr²⁺, Pb²⁺, Cu²⁺, Cd²⁺, Zn²⁺, Ni²⁺, and Co²⁺.

In particular aspects, the mucoadhesive gel formulation comprises one or more of alginate (Alg), polyethylene oxide (PEO), methacrylic acid, methyl methacrylate (E), hydroxypropylcellulose (HPC), and carboxymethyl cellulose (CMC).

In certain aspects, the mucoadhesive formulation further comprises one or more surfactants. In particular aspects, the one or more surfactants are selected from the group consisting of polyoxyethylene sorbitol ester and sorbitan oleate.

In particular aspects, the mucoadhesive gel comprises a formulation selected form the group consisting of 1% w/v sodium alginate, 1.5% w/v sodium alginate, 2.5% w/v sodium alginate, 1% w/v PEO, 2.5% w/v PEO, Alg2.5:PEO1=1:1 with 1% w/v polyoxyethylene sorbitol ester, Alg2.5:PEO1=1:1 with 0.5% w/v polyoxyethylene sorbitol ester, Alg2.5:PEO1=1:1 with 0.5% w/v Sorbitan oleate, Alg2.5: methacrylic acid, and methyl methacrylate=1:1, Alg2.5: methacrylic acid, and methyl methacrylate=1:1, Alg2: methacrylic acid, and methyl methacrylate=3:1, 1.5% w/v sodium alginate, 1% w/v methacrylic acid, and methyl methacrylate, 1.2% w/v sodium alginate, 0.8% w/v methacrylic acid, and methyl methacrylate, 1.5% w/v sodium alginate, 0.1% w/v sodium carboxymethyl cellulose, 1.5% w/v sodium alginate, 0.5% w/v sodium carboxymethyl cellulose, 1.5% w/v sodium alginate, 1% w/v sodium carboxymethyl cellulose, 1.5% w/v sodium alginate, 2.5% w/v PEO, 2% w/v hydroxypropylcellulose, and 4% w/v hydroxypropylcellulose.

In more particular aspects, the mucoadhesive gel formulation comprises 1.5% w/v sodium alginate and 1 M CaCl₂. In yet more particular aspects, the mucoadhesive gel formulation comprises 1.2% w/v sodium alginate, 0.8% w/v methacrylic acid, 0.8% w/v methyl methacrylate, and 1 M CaCl₂. In even yet more particular aspects, the mucoadhesive gel formulation comprises 1.2% w/v sodium alginate, 0.8% w/v methacrylic acid, and 0.8% w/v methyl methacrylate, 0.5% w/v polyoxyethylene sorbitol, a buffer, 0.9% w/v NaCl, a dye, ascorbic acid, 1 M CaCl₂, and water.

In certain aspects, the mucoadhesive gel formulation further comprises 0.01 mg/mL of epinephrine or an epinephrine-containing nanoparticle. In more certain aspects, the epinephrine comprises free epinephrine or in an epinephrine-containing nanoparticle or a combination of both.

In certain aspects, the mucoadhesive gel formulation comprises 1.2% w/v sodium alginate, 0.8% w/v methacrylic acid, 0.8% w/v methyl methacrylate, 0.5% w/v PEO, 0.5% w/v polyoxyethylene sorbitol, a buffer, 0.9% w/v NaCl, a dye, ascorbic acid, and 1 M CaCl₂, and water.

In certain aspects, the presently disclosed formulations comprise one or more additional therapeutic agents. In particular aspects, the one or more local anesthetics is lidocaine.

In other aspects, the presently disclosed subject matter provides a method for preventing or controlling a gastrointestinal bleed, the method comprising administering to a subject in need of treatment thereof a presently disclosed injectable formulation, a presently disclosed mucoadhesive formulation, or combinations thereof.

In some aspects, the gastrointestinal bleed is associated with a deep source of gastrointestinal bleeding, e.g., an ulcer, and the formulation comprises a presently disclosed injectable formulation. In certain aspects, the presently disclosed formulation further comprises one or more dyes to maximize visibility of deep blood vessels when the formulation is injected into GI tissue. In other aspects, the presently disclosed formulation comprise one or more excipients, one or more buffers, one or more salts, and combinations thereof.

In other aspects, the gastrointestinal bleed is associated with a superficial source of gastrointestinal bleeding, e.g., a cancerous lesion, and the formulation is a presently disclosed mucoadhesive formulation.

In yet other aspects, the presently disclosed subject matter provides a method for separating diseased tissue from normal tissue providing a sub-mucosal cushion, the method comprising: (a) injecting a presently disclosed injectable composition under the diseased tissue to form a depot thereunder, thereby lifting the diseased tissue from the normal tissue; and (b) dissecting the diseased tissue to separate the diseased tissue from the normal tissue at a dissection site. In certain aspects, the diseased tissue comprises a polyp. In certain aspects, the presently disclosed formulations further comprise one or more electrolytes, e.g., one or more salts, to ensure conductance of electricity during cautery-based dissection of the diseased tissue after lifting.

In more certain aspects, the method further comprises an endoscopic procedure selected from the group consisting of endoscopic mucosal resection (EMR), endoscopic sub mucosal dissection (ESD), endoscopic myotomy, third-space endoscopy, endoscopic tunneling, and combinations thereof. In further aspects, the method further comprises administering a presently disclosed mucoadhesive formulation to the dissection site to prevent or control bleeding thereof. In further aspects the mucoadhesive formulation bonds firmly with the sub-mucosa at the site of mucosal resection or polypectomy. In some aspects, the mucoadhesive formulation thereafter shrinks or contracts, thereby approximating the margins of tissue defect and provide some tamponade effect.

In other aspects, the presently disclosed subject matter provides an endoscopic injection needle for delivering an injectable solution comprising a mixture of at least two formulations to a tissue treatment site, the endoscopic injection needle comprising:

(a) a connecter comprising a proximal portion and a distal portion: (i) at least two inlet ports at the proximal portion of the connecter, wherein the at least two inlet ports are in fluid communication with a reservoir; (ii) an outlet port at the distal portion of the connector, wherein the outlet port is in fluid communication with the reservoir; and (iii) a plunger movably positionable within the proximal portion of the reservoir, the plunger providing a seal at the proximal portion of the connector to prevent the injectable solution from flowing out of the proximal portion of the connector and wherein the plunger further comprises a plunger advancing member configured to force the injectable solution from the reservoir through the outlet port at the distal portion of the connector;

(b) a static mixing chamber comprising a proximal portion and a distal portion, wherein the proximal portion of the static mixing chamber is in fluid communication with the outlet port at the distal portion of the reservoir, wherein the static mixing chamber is configured to receive the injectable solution from the reservoir; and

(c) a sheath comprising a proximal portion and a distal portion, wherein the proximal portion of the sheath is in fluid communication with the distal portion of the static mixing chamber, and wherein the sheath further comprises a needle enclosed therein, wherein the distal portion of the sheath is movable to expose the needle for insertion into the tissue treatment site.

In other aspects, the presently disclosed subject matter provides an endoscopic injection needle for delivering an injectable mucoadhesive gel formulation to a tissue treatment site, the endoscopic injection needle comprising:

(a) at least two inlet ports, wherein a first inlet port is in fluid communication with a first chamber for accommodating a mucoadhesive gel and a second inlet port is in fluid communication with a second chamber for accommodating an activator;

(b) at least two outlet channels, wherein a first outlet channel is in fluid communication with the first chamber and a second outlet channel is in fluid communication with the second chamber; and

(c) a first plunger and a second plunger movably positionable within a proximal portion of the first chamber and a proximal portion of the second chamber, the first and second plunger providing a seal at the proximal portion of the first and second chamber to prevent the mucoadhesive gel injectable solution from flowing out of the proximal portion of the first chamber and the activator from flowing out of the proximal portion of the second chamber, wherein the first plunger and the second plunger further comprise a single plunger advancing member configured to force the mucoadhesive gel from the first chamber through the first outlet channel and the activator from the second chamber through the second outlet channel,

wherein the plunger and seal of the second chamber are operationally positioned to form a gap to delay delivery of the activator in relation to delivery of the mucoadhesive gel to the tissue treatment site.

In some aspects, the presently disclosed subject matter provides a kit comprising at least one of presently disclosed injectable formulation, a presently disclosed mucoadhesive formulation, or combinations thereof. In certain aspects, the kit further comprises at least one of the endoscopic injection needles disclosed herein.

In other aspects, the presently disclosed subject matter provides a method for delivering one or more therapeutic agents to a targeted site in a gastrointestinal (GI) tract, the method comprising administering a presently disclosed formulation with endoscopy to the targeted site.

In certain aspects, the one or more therapeutic agents are selected from the group consisting of one or more corticosteroids, one or more antibiotics, one or more chemotherapeutic agents, one or more tumor necrosis factor inhibitors, one or more angiogenesis inhibitors, one or more kinase inhibitors, one or more immunosuppressive agents, one or more 5-aminosalicylic acid (5-ASA) agents, polytetrafluoroethylene, one or more silicone-based gels, polyacrylamide, polyacrylonitrile, and combinations thereof.

In certain aspects, the presently disclosed method further comprises treating or preventing one or more diseases, disorders, or conditions selected from the group consisting of one or more strictures in an esophagus or intestine, one or more infected collections around a GI tract, dysmotility or incontinence, inflammatory bowel disease (IBD) and related inflammation, one or more fistulae, and inflammation in liver, pancreas, stomach, intestine, and combinations thereof.

In other aspects, the presently disclosed subject matter provides a method for sealing a perforation in tissue of a GI tract, the method comprising administering a presently disclosed mucoadhesive gel formulation to the perforated tissue.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 shows images of gastric ulcer bleeding and injection of a presently disclosed formulation comprising epinephrine in an ulcer base using an endoscope-based injection needle;

FIG. 2 is an image of a cancerous lesion with surface bleeding and topical application of a presently disclosed epinephrine mucoadhesive gel for superficial bleed using an endoscope-based needle or catheter;

FIG. 3A, FIG. 3B, FIG. 3C, FIG. 3D, FIG. 3E, and FIG. 3F are graphs showing the viscosity as a function of temperature and shear rate vs shear stress as evaluated using rheometer. FIG. 3A and FIG. 3B show the viscosity as a function of temperature of representative polymers demonstrating reverse thermal gelation i.e., as the temperature increases the viscosity increases. FIG. 3C, FIG. 3D, FIG. 3E, and FIG. 3F graphs the rheological properties of the representative polymers as a function of shear rate and shear stress. Gel polymers could be screened for those displaying Newtonian behavior and hysteresis;

FIG. 4A, FIG. 4B, and FIG. 4C show (FIG. 4A) a Hanson vertical diffusion cell used to perform the epinephrine release studies. FIG. 4B graphs the cumulative release of epinephrine in three representative gel formulations and demonstrates how epinephrine could permeate across the mucosa for local effect. FIG. 4C graphs the cumulative release of epinephrine from representative formulations containing nanoparticles; compared to FIG. 4B the nanoparticles greatly extended the duration of release of epinephrine;

FIG. 5A and FIG. 5B show an ex-vivo experiment using a pig stomach. FIG. 5A, to evaluate the feasibility of injection of an epinephrine gel using an endoscopy injection needle. Epinephrine gel depots could be created. Cushion height could be measured. FIG. 5B shows dissection of the gel injection demonstrating intact depot;

FIG. 6A. FIG. 6B, FIG. 6C, FIG. 6D, and FIG. 6E show an in-vivo experiment with injection of gel polymers into a pig stomach using endoscopy (FIG. 6A, FIG. 6B). Endoscopy view of stomach after gel injection (FIG. 6C). Autopsy examination of gel depot in pig stomach after 72 hours (FIG. 6D). Microscopic examination at the injection site in the stomach demonstrating no adverse reaction (FIG. 6E);

FIG. 7A. FIG. 7B, FIG. 7C show an in-vivo experiment with injection of epinephrine containing gel into pig stomach to control bleeding from an ulcer. Endoscopy view of bleeding ulcer in pig stomach (FIG. 7A) and on injection of representative gel containing epinephrine nanoparticle (FIG. 7B), the bleeding resolves (FIG. 7C);

FIG. 8 is an ex-vivo experiment demonstrating the application of a representative gel formulation followed by a chemical activator to pig stomach tissue resulting in firm adhesion;

FIG. 9A. FIG. 9B, FIG. 9C, FIG. 9D and FIG. 9E show an in-vivo experiment with the application of epinephrine containing mucoadhesive gel to bleeding ulcers in pig stomach. Endoscopy view of bleeding ulcer in pig stomach (FIG. 9A) and on application of representative mucoadhesive gel containing epinephrine (FIG. 9B), followed by chemical activator (FIG. 9C), a firm adhesive gel layer forms on the surface of the ulcer and the bleeding resolves (FIG. 9D). On flushing with a jet of water the gel layer does not wash off (FIG. 9E) and the adhesive gel can be seen on necropsy after 24 hours (FIG. 9F);

FIG. 10A. FIG. 10B, FIG. 10C and FIG. 10D show an ex-vivo experiment with the application of mucoadhesive gel to seal perforation in pig stomach tissue. This perforation mimics a perforation that can result as a complication of polypectomy during endoscopy. FIG. 10A shows a pig stomach with 3-mm hole and leak when water is filled in the stomach (FIG. 10B). After application of the mucoadhesive gel (FIG. 10C) over the perforation site, the defect is sealed and no longer has a leak when water is filled (FIG. 10D);

FIG. 11 is a schematic representation of the use of epinephrine injectable gel and epinephrine mucoadhesive gel during polyp removal using the endoscopic mucosal resection (EMR) technique. These diagrams showing injection of a lifting gel solution during colon polyp removal with endoscopy solution to separate gastrointestinal wall layers (FIG. 11A); polyp resection with endoscopy snare and often using electro cautery (FIG. 11B); bleeding following lifting and polyp removal (FIG. 11C); application of mucoadhesive gel at the site of polypectomy (FIG. 11D) followed by activator spray (FIG. 11E) for bleeding control or prevention; shrinking of mucoadhesive gel causing proximation of borders and tamponade effect (FIG. 11F);

FIG. 12 shows a modified endoscopy injection needle to provide varying combinations of gel polymers or activators to customize epinephrine gel properties according to the indication (duration of action needed, mucoadhesive vs. sustained release properties); and

FIG. 13 shows a modified double channel endoscopy catheter with a wide channel for mucoadhesive gel and a narrow channel for the activator. There are separate chambers for mucoadhesive gel and the activator to keep them separated but a single plunger flare for ease of use during the delivery. In order to create a delay between the application of mucoadhesive gel and the activator, a gap is created between the plunger and the seal.

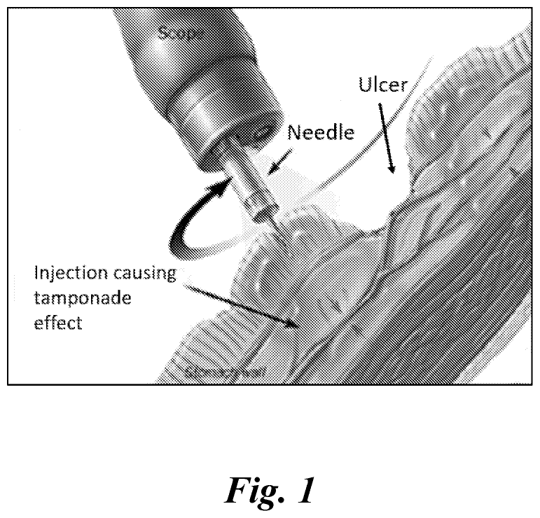

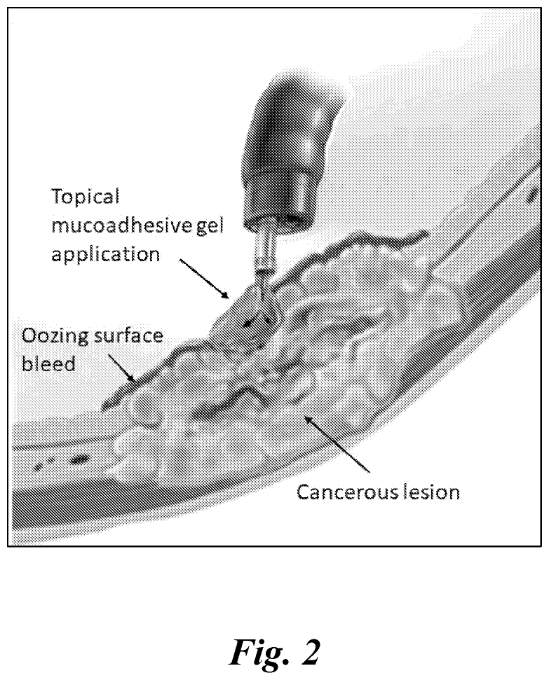

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Gel for Use in Gastrointestinal Endoscopy and Endodermal, Epidermal, and Other Mucosal Uses

Epinephrine has innumerable applications and has been extensively studied since it was first extracted at The Johns Hopkins University in 1897. Abel and Crawford, 1897. The use of epinephrine in the GI space, however, has essentially remained unchanged in the past 40 years. Epinephrine has numerous applications in GI endoscopy, especially for the control of GI bleeding and to ensure a clean operative field during endoscopic interventions. The use of epinephrine, however, has been limited by the short duration of action due to its physical properties and pharmacokinetics.

Epinephrine is typically used in a 1:10,000 to 1:20,000 dilution ratio, with the volume being less than about 10 mL. Park et al., 2004. Larger volumes of diluted epinephrine have been shown to be more efficacious in reducing re-bleeding due to an improved tamponade affect, but due to its low viscosity, it is challenging to inject larger volumes of epinephrine effectively. A higher concentration of epinephrine has been shown to be superior in hemostasis, Sarmento et al., but a sudden increase in blood levels of epinephrine from a large volume of a high-concentration epinephrine injection can cause systemic side effects.

Further, the current treatment modalities to control GI bleed have limited efficacy, limited applicability, or are exorbitantly expensive. Although current treatment modalities can control acute GI bleed in most situations, they only provide temporary control of the acute bleed with a high recurrence of bleeding afterward. Giday et al., 2011; Sung et al., 2011. Another major limitation of current treatment modalities is the endoscopy view is obscured by a thick opaque layer formed by the hemostatic agent, thereby preventing any further endoscopic intervention during the same session. Other hemostatic agents known in the art have been found to be ineffective for GI use. Lee et al., 2017.

An unmet need for the presently disclosed formulation was further recognized from previous research involving the use of epinephrine to prevent endoscopy-related pancreatitis, including a multi-center, randomized trial involving 960 patients. Akshintala et al., 2013; Kamal et al., 2019. In those studies, it was observed that the efficacy of epinephrine for preventing endoscopy-related pancreatitis was limited due to its short half-life. Without wishing to be bound to any one particular theory, it is thought that the presently disclosed long-acting, viscous, sustained-release epinephrine formulation will effectively control GI bleeding without the need for a second therapy as recommended by the ASGE.

Accordingly, the presently disclosed subject matter provides a combination of epinephrine with biomaterials/gel polymers, which allow mucoadhesion and creation of injectable gel depot systems for the sustained release of epinephrine. More particularly, in some embodiments, the presently disclosed subject matter provides a sustained-release epinephrine formulation to effectively control GI bleeding as a stand-alone therapy. In some embodiments, the presently disclosed injectable gel formulation can be used to treat a deep source of GI bleeding, such as an ulcer. In other embodiments, the presently disclosed mucoadhesive gel formulation can be used to treat a superficial source of bleeding, such as an oozing cancerous lesion.

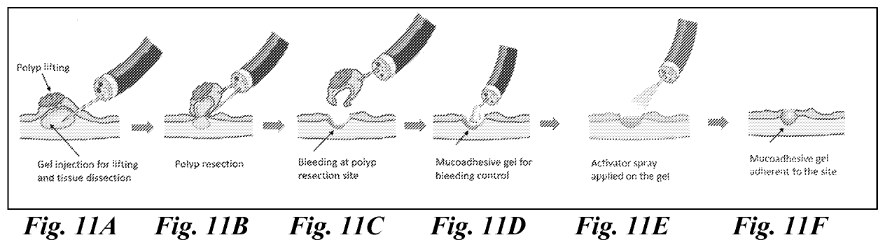

In other embodiments, the presently disclosed sustained-release epinephrine formulation can be used to facilitate endoscopy interventions for separating diseased tissue from normal tissue by lifting and dissection, such as in the lifting and dissection of large polyps and in endoscopic tunneling procedures (see, e.g., FIG. 11). The tissue lifting/dissection principle is applicable in a multitude of other GI applications including, but not limited to, endoscopic mucosal resection (EMR) (FIG. 11), endoscopic sub mucosal dissection (ESD), endoscopic myotomy, third-space endoscopy, and the like. In yet other embodiments, the presently disclosed sustained-release epinephrine formulation can prevent bleeding after dissection of diseased tissue, circumventing the need for hemostat clips. In particular embodiments, the presently disclosed injectable epinephrine gel, which has a high viscosity, is amenable to the lifting/dissection procedure.

Further, the tissue defect created by dissection causes immediate or delayed bleeding and requires the placement of a hemostat clip, which is an expensive procedure. Mohan et al., 2019. The presently disclosed mucoadhesive epinephrine gel, when applied on the dissection site avoids the need for a hemostat clip. The EMR technique is increasingly being used and an estimated 600,000 such procedures are annually performed in US. Ju et al., 2020. Currently available products for use in lifting and tissue dissection are limited by their inability to control bleeding. It is not possible to mix epinephrine in gels known in the art, which is a significant limitation to their use. When used together, the presently disclosed epinephrine gels can be used to both lift and dissect polyps (injectable formulation) and control and/or prevent bleeding (mucoadhesive formulation).

The presently disclosed epinephrine gels are expected to be attractive to endoscopy units globally since these gels can be used to treat a majority of GI bleed conditions and be used for tissue lifting or dissection and prevention of bleed after dissection. The dual purpose of the presently disclosed epinephrine gels will essentially replace the need for multiple separate, expensive endoscopy accessories.

The presently disclosed epinephrine gel system will lead to better efficacy for epinephrine in several medical application and has several advantages over traditional delivery stems. Such advantages include, but are not limited to the following:

The presently disclosed subject matter is a targeted delivery system, which allows for an increased concentration of epinephrine at the target site while reducing the side effects of epinephrine to off-target tissue and organs.

The presently disclosed subject matter undergoes in-situ gel formation, which enables the formulation to be delivered as a low-viscous liquid using conventional, currently available endoscopy injection accessories.

The release from the presently disclosed gel depot has an initial burst release to stop acute bleeding followed by long-term release for at least 72 hours to treat long-term bleeding and/or to prevent delayed bleeding.

The presently disclosed gel depot is resorbed by the body and does not require surgical removal.

The presently disclosed mucoadhesive formulation binds firmly with submucosa at the site of polyp removal or mucosal resection and shrinks or contracts, thereby approximating the margins of tissue defect and providing some tamponade, bleeding control. The presently disclosed injectable epinephrine gel formulation and mucoadhesive epinephrine gel formulation have several potential applications in gastroenterology and other medical specialties.

Beyond application to GI bleeding, the presently disclosed epinephrine gels are expected to control bleeding at other mucosal surfaces and can be used for dental, pulmonary, otolaryngology, gynecological applications, and the like, where short acting epinephrine formulations are currently being used.

### I.A. Injectable and Mucoadhesive Gel Formulations for the Sustained Release of Epinephrine

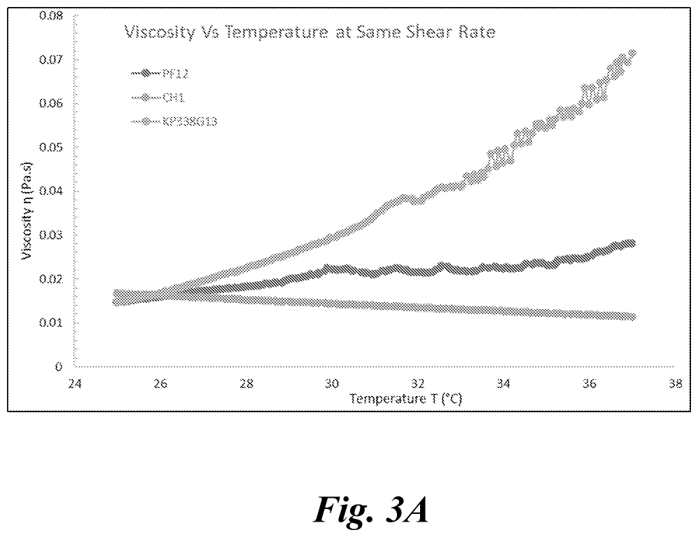

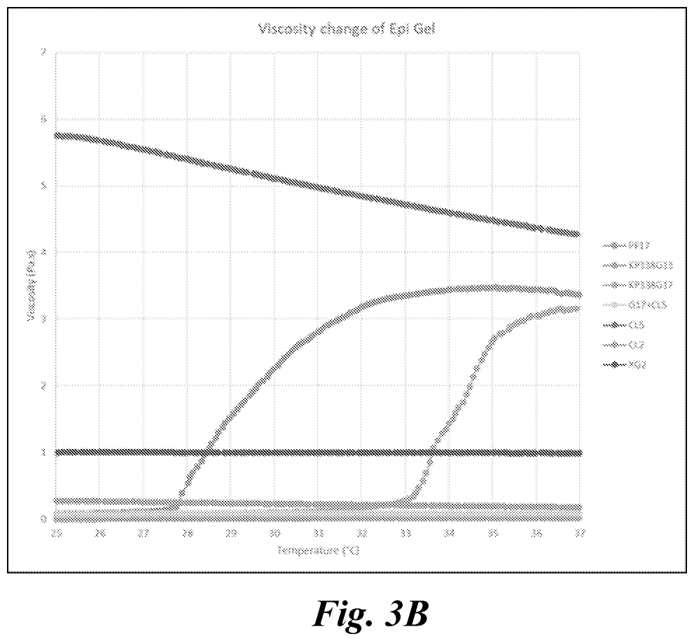

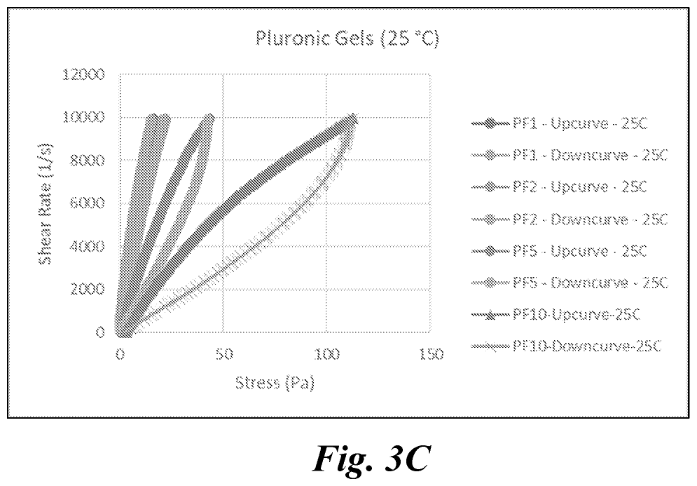

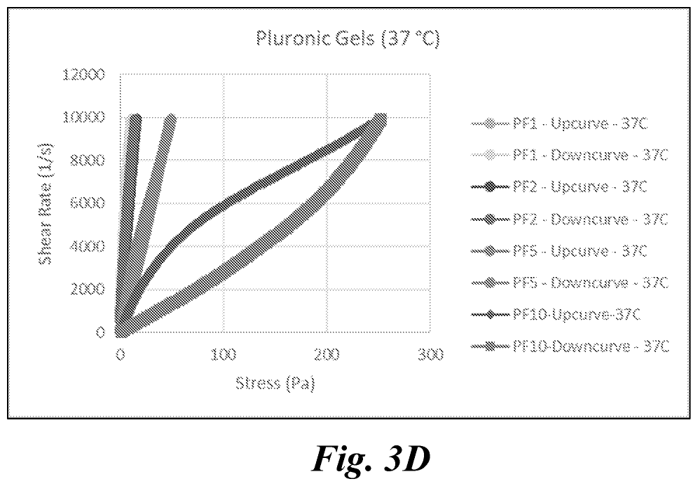

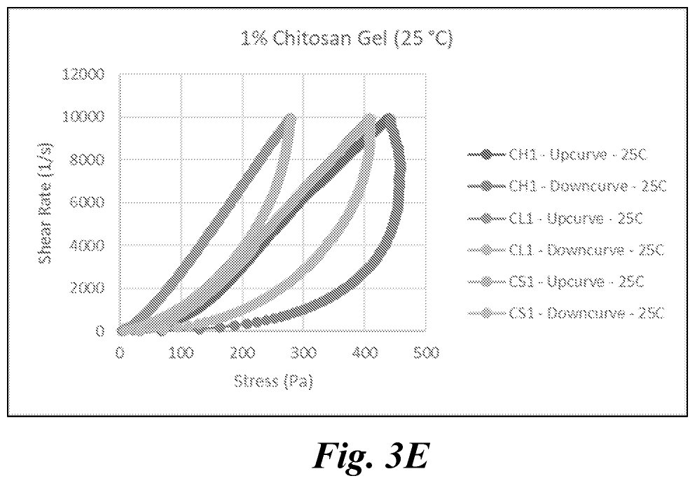

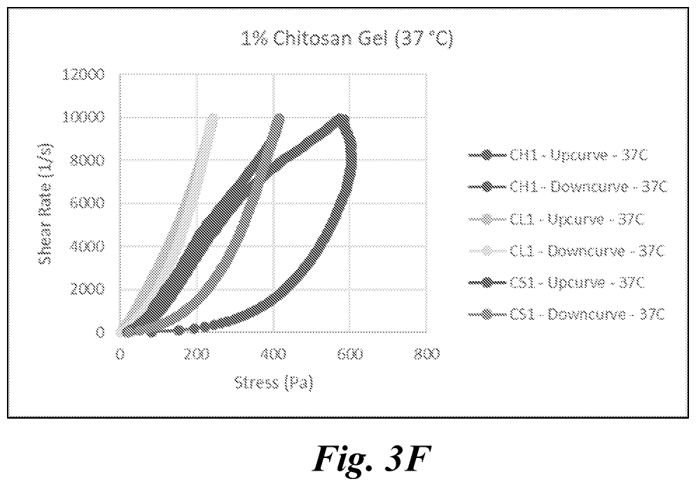

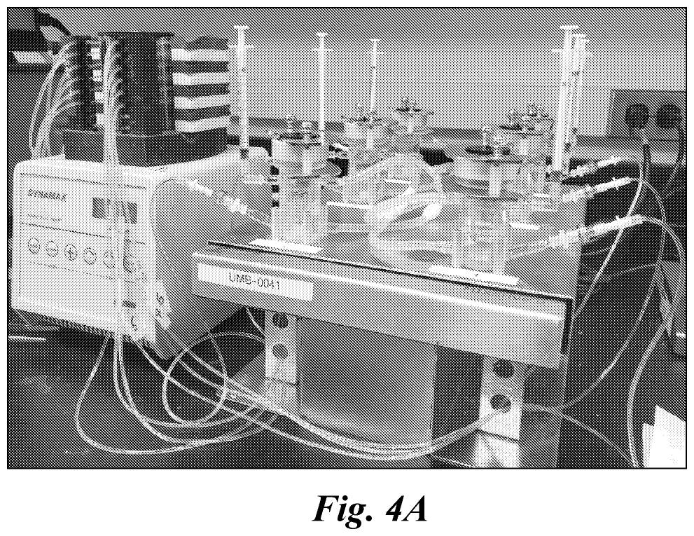

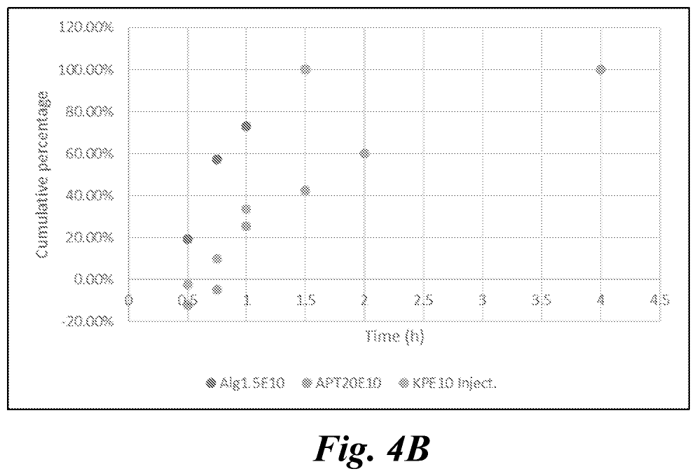

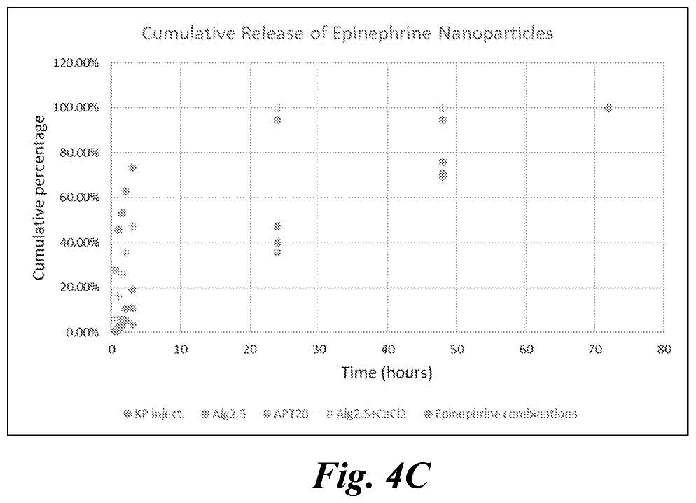

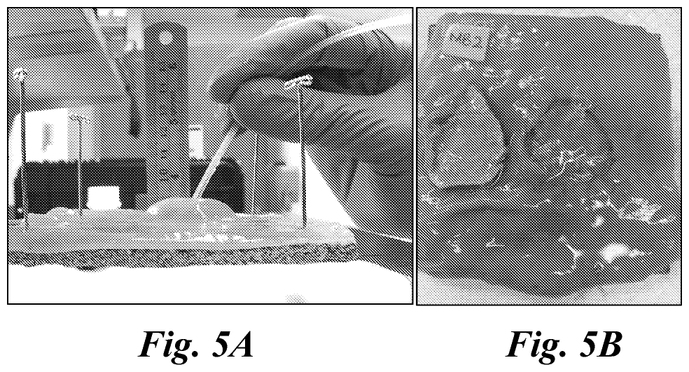

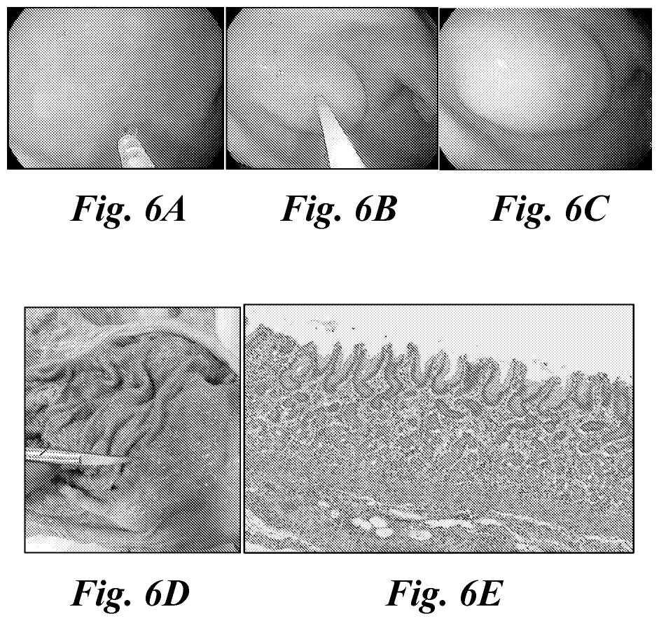

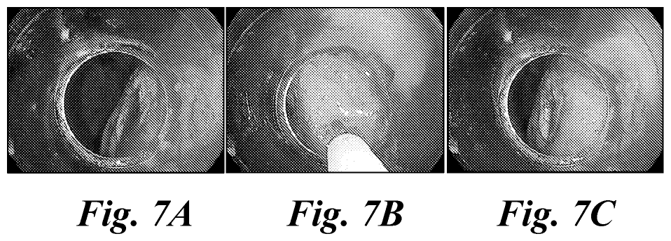

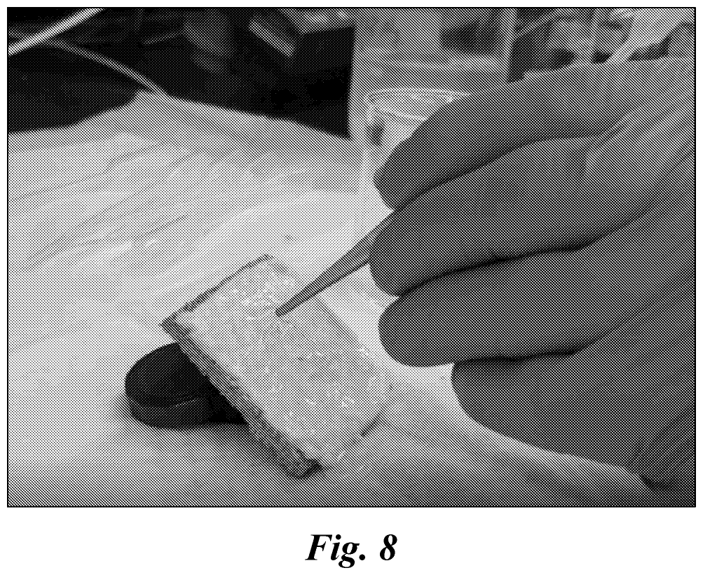

A wide array of gel polymer families with varying properties was investigated to identify the most suitable combination of constituents and the optimum concentration of epinephrine to generate the desired properties of a long-acting epinephrine gel. These investigations include a series of in-vitro (FIG. 3., FIG. 4), ex-vivo (FIG. 5, FIG. 8) and in-vivo animal experiments (FIG. 6, FIG. 7, FIG. 9). The gel polymers were modified iteratively to maximize the timeframe for epinephrine release based on experiments in Hanson diffusion cells (FIG. 4B, FIG. 4C). The feasibility of injecting these polymers into GI tissue was tested using ex-vivo experiments (FIG. 5A). To test their use with endoscopy and to evaluate safety, gel polymers containing epinephrine were injected into a live pig stomach at different sites (FIG. 6). Based on these studies, two separate gel formulations, an injectable formulation and a mucoadhesive formulation, each having distinct properties to cater to the applications of interest to endoscopists, were developed.

### I.A.1. Injectable Gel Formulation for the Sustained Release of Epinephrine

In some embodiments, the presently disclosed subject matter provides a long-acting injectable epinephrine gel depot also for control of GI bleeding and for facilitating GI endoscopic interventions. Accordingly, in some embodiments, the presently disclosed subject matter provides an injectable gel formulation for the sustained release of epinephrine, the formulation comprising epinephrine, or a pharmaceutically acceptable salt thereof, and a degradable polymer or a combination of polymers.

In some embodiments, the injectable gel formulation further comprises from about 0.001 mg/mL to about 0.1 mg/mL epinephrine, including 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.10 mg/mL, either immediate release or an epinephrine-containing nanoparticle or a combination of both immediate release and an epinephrine-containing nanoparticle.

As used herein the term “biocompatible” refers to an ability to be in contact with a living system without producing an adverse effect. The term “biodegradable” refers to the capability of being degraded by one or more biological activities or functions. Such degradation can result, for example, from enzymatic degradation in vivo. In other embodiments, the degradation can occur via hydrolysis, which is not necessarily a biologic process, in which the term is “degradable” or “hydrolytic degradation.” Other degradation can occur, for example, through one or more physical processes, such as flaking, peeling, or shedding, in which the degradable polymer is otherwise removed from the GI tract.

Degradable polymers known in the art include polyesters including, but not limited to, poly(glycolic acid) (PGA), poly(D,L-lactic acid) (PLA), poly(D,L-lactic-co-glycolic acid) (PLGA), and poly(caprolactone) (PCL); PLGA copolymers, such as block copolymers of PLGA and PEG (PLGA-PEG), including PLGA-PEG deblocks; poly(ortho esters) (POEs) including, but not limited to POE I, POE II, POE III, and POE IV; poly(anhydrides); poly(amides); poly(ester amides); poly(phosphoesters); poly(alkyl cyanoacrylates); and natural degradable polymers, such as collagen, albumin, gelatin, and polysaccharides, such as agarose, alginate, carrageenan, hyaluronic acid (HA), dextran, chitosan, and cyclodextrins.

More particularly, in some embodiments, the degradable polymer is a synthetic polymer selected from the group consisting of poly(lactic acid) (PLA), poly(DL-lactide), poly(dl-lactic acid), poly(DL-lactide-co-glycolide), poly(lactic-co-glycolic acid) (PLGA), poly(caprolactone) (PCL), poly(E-caprolactone), poly(P-dioxanone), poly(hydroxybutyrate), poly(B-malic acid), poloxamer, polycarbophil and Ca++ salt, poly(methyl vinyl ether/maleic anhydride), polyanhydrides, polyphosphazenes, poly(ortho esters), poly(phosphoester), polyhydroxyalkanoates (PHA), polyurethane (PUR), carbomer, and cyclomethicone. In other embodiments, the degradable polymer is a natural polymer selected from the group consisting of alginic acid, Ca alginate and Na salt, agar, xanthan gum, chitosan, chitin, guar gum, a carrageenan, gellan gum, starch modified, silk protein polymers, elastine protein polymers, silk-elastin protein polymers, collagen, hyaluronic acid, pseudo-amino acids, albumin, fibrinogen, maltodextrin, and gelatin.

As used herein, the term “poloxamer” refers to nonionic triblock copolymers comprising a central hydrophobic chain of polyoxypropylene (poly(propylene oxide)) flanked by two hydrophilic chains of polyoxyethylene (poly(ethylene oxide)) of the general formula of:

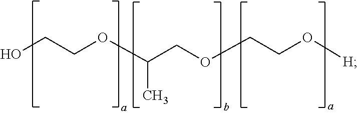

wherein each a is independently an integer from 2 to 130 and b is an integer from 15 to 67. Such copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mol} and a 70% polyoxyethylene content).

In some embodiments, the formulation further comprises one or more excipients including, but not limited to, waxes such as carbuna wax, Egg Phospholipids including (Dilauroyl phosphatidylcholine, Dimyristoyl phosphatidylcholine, Dipalmitoyl phosphatidylcholine, Distearoyl phosphatidylcholine, Dioleoyl phosphatidylcholine, Dioctanoyl phosphatidylcholine, Dierucoyl phosphatidylcholine, Palmitoyloleoyl phosphatidylcholine, Dimyristoyl phosphatidylglycerol, sodium salt, Dipalmitoyl phosphatidylglycerol, sodium salt, Distearoyl phosphatidylglycerol, sodium salt, Dioleoyl phosphatidylglycerol, sodium salt, Palmitoyloleoyl phosphatidylglycerol, sodium salt, Dimyristoyl phosphatidylethanolamine, Dipalmitoyl phosphatidylethanolamine, Distearoyl phosphatidylethanolamine, Dioleoyl phosphatidylethanolamine, Dimyristoyl phosphatidic acid, sodium salt, Dipalmitoyl phosphatidic acid, sodium salt, Distearoyl phosphatidic acid, sodium salt, Dioleoyl phosphatidylserine, sodium salt), glyceryl monooleate, lecithin, oleic acid, dibutyl sebacate salts, such as NaCl, preservatives, glycerin, chlorhexidine, dimethyl sulfoxide, glyceryl behenate, Na stearate, glyceryl palmitostearate, olive oil, and sucrose stearate.

The presently disclosed formulations also include buffers, such as Ascorbic acid, Maleic acid, Tartaric acid, Lactic acid, Citric acid, Acetic acid, Sodium bicarbonate, Sodium phosphate, and electrolytes, such as NaCl, KCl, Na₂PO₄, CaPO₄, CaCl₂, and Na Lactate.

In more particular embodiments, the degradable polymer is a diblock copolymer comprising monomethoxy poly(ethylene glycol)-block-poly(D,L-lactide) (mPEG-PDLLA). A depot created with the presently disclosed injectable formulation is expected to release epinephrine over a time period of up to 192 hours (not less than 72 hours) with a low burst effect (around 7% in the first 8 h). This formulation can be used to control GI bleeding from ulcers that require injection and lifting of large polyps (FIG. 7). In some embodiments, the injectable formulation has a high viscosity. For example, the injectable formulation can have a viscosity ranging from about 0.01 cp to 3000 cp, including 0.01, 0.1, 1, 5, 10, 50, 100, 200, 300, 400, 500, 600, 700, 800, 900, 1000, 1500, 2000, 2,500, and 3000 cp. In some embodiments, the viscosity has a range from about 10 to about 100 cp, including 10, 20, 30, 40, 50, 60, 70, 80, 90, and 100 cp. In yet other embodiments, the viscosity has a range from about 8 to about 10 cp, including 8.0, 8.5, 9.0, 9.5, and 10 cp. In other embodiments, the injectable formulation has a low concentration of epinephrine. In yet other embodiments, the injectable formulation has a slow release of epinephrine.

The presently disclosed injectable formulation can be used in isolation or in combination with a solvent for the creation of a gel depot with the properties of interest pertaining to drug diffusability and availability. Such depot gel prolongs the bioavailability of epinephrine and increases the duration of action in controlling the mucosal/submucosal bleed in GI tissue. This gel depot system also prolongs the tamponade affect thereby providing additional advantage in controlling bleed. The depot epinephrine gel system can be delivered using endoscopic accessories currently available, to the area of interest. The gel formulation flows in between tissue spaces in sub-mucosa and has auto-dissection property.

Indocyanine green, methylene blue, indigo carmine or other dyes can be added to the formulation to maximize visibility of deep blood vessels when injected into GI tissue. Salts (sodium chloride equivalents) also can be added to ensure conductance of electricity during cautery-based dissection of the diseased tissue after lifting.

### I.A.2. Mucoadhesive Gel Formulation for the Sustained Release of Epinephrine

In embodiments, the presently disclosed subject matter provides a long-acting mucoadhesive epinephrine gel formulation or composition for controlling GI bleeding and for facilitating GI endoscopic interventions. In certain embodiments, the presently disclosed subject matter provides a mucoadhesive gel formulation for the sustained release of epinephrine, the formulation comprising epinephrine, a degradable polymer, and a mucoadhesive coating.

More particularly, in some embodiments, the degradable polymer is a synthetic polymer selected from the group consisting of poly(lactic acid) (PLA), poly(DL-lactide), poly(dl-lactic acid), poly(DL-lactide-co-glycolide), poly(lactic-co-glycolic acid) (PLGA), poly(caprolactone) (PCL), poly(E-caprolactone), poly(P-dioxanone), poly(hydroxybutyrate), poly(B-malic acid), poloxamer, polycarbophil and Ca++ salt, poly(methyl vinyl ether/maleic anhydride), polyanhydrides, polyphosphazenes, poly(ortho esters), poly(phosphoester), polyhydroxyalkanoates (PHA), polyurethane (PUR), carbomer, and cyclomethicone.

In other embodiments, the degradable polymer is a natural polymer selected from the group consisting of alginic acid, Ca alginate and Na salt, agar, xanthan gum, chitosan, chitin, guar gum, a carrageenan, gellan gum, starch modified, silk protein polymers, elastine protein polymers, silk-elastin protein polymers, collagen, hyaluronic acid, pseudo-amino acids, albumin, fibrinogen, maltodextrin, and gelatin.

The mucoadhesive polymers have hydrophilic and hydrophobic components. The hydrophobic component may comprise synthetic hydrophobic polymers such as, but not limited to, polyesters, polyurethanes, polyureas, polycarbonates, polyethers, polysulfides, polysulfonates, polyimides, polybenzimidazoles, and combinations thereof. The hydrophobic polymer may also be a naturally occurring hydrophobic polymer such as a lipoglycan, a proteoglycan, and the like, modified versions thereof, or combinations thereof. Examples of hydrophobic polymers for inclusion in the present mucoadhesive formulation, thus, include, but are not limited to, a polylactide, polyglycolide, poly(lactide-co-glycolide, poly(e-caprolactone), poly hydroxybutyrate, poly(dioxanone), poly(3-hydroxybutyrate), poly(3-hydroxyval crate), poly(valcrolactone), poly(tartonic acid), poly(malonic acid), poly(anhydrides), poly(orthoesters), polyphosphazenes and acryloyloxy dimethyl-y-butyrolactone (DBA) and other lactone-containing polymers, and combinations thereof.

The hydrophilic polymers include but are not limited to, polyacrylic acids, polyalcohols, polyacrylates, polyurethanes, polyacrylamines, polyacrylamides, polyethers and polypyrollidones. Thus, suitable hydrophilic polymers may include those comprising one or more monomers selected from acrylate, acrylic acid, methacrylate, methacrylic acid, methyl acrylate, ethyl acrylate, methyl methacrylate, acrylonitrile, 2-chloroethyl vinyl ether, 2-ethylhexyl acrylate, hydroxyethyl methacrylate, butyl acrylate, butyl methacrylate, trimethylolpropane triacrylate, hydroxypropylmethacrylamide, hydroxyethyl acrylate, poly(ethylene glycol) methacrylate, poly(N-isopropylacrylamide) (RNGRAM), poly(vinyl alcohol) (PVA), poly(2-oxazoline), polyethylene glycol, or polyvinylpyrollidone polymers, or copolymers thereof.

In some embodiments, the mucoadhesive component is capable of binding to mucin and sub-mucosa. In this regard, the mucoadhesive will be selected to bind to cis-diol groups present in carbohydrates within mucin, e.g., sialic acids, N-acetylglucosamine, N-acetylgalactosamine, galactose and fucose. Examples of a suitable mucoadhesive for this purpose, include, but are not limited to, boronic acids such as phenylboronic acid, 2-thienylboronic acid, methylboronic acid, cis-propenylboronic acid, trans-propenylboronic acid, (4-allylaminocarbonyl)benzene-boronic acid, (4-aminosulfonylphenyl)boronic acid, (4-benzyloxy-2-formyl)phenyl-boronic acid, (4-hydroxy-2-methyl)phenylboronic acid, (4-hydroxy-2-methyl)phenyl-boronic acid, (4-methanesulfonylaminomethyl-phenyl)boronic acid, (4-ethane-sulfonylamino-methylphenyl)boronic acid, (4-methylaminosulfonylphenyl)boronic acid, (4-methylaminosulfonylphenyl)boronic acid, (4-phenylaminocarbonyl-phenyl)boronic acid, (4-henylaminocarbonyl-phenyl)boronic acid, (4-sec-butyl)benzeneboronic acid, (2,6-dimethoxy-4-methylphenyl)boronic acid, (2,6-dimethoxy-4-methylphenyl)-boronic acid, (2-methylpropyl)boronic acid, (2-methylpropyl) boronic acid,(3-acetamido-5-carboxy)phenylboronic acid, (3-acetamido-5-carboxy) phenyl boronic acid, (3-acetamidomethylphenyl)boronic acid, (3-acetamidomethylphenyl) boronic acid, (3-allylaminocarbonyl)benzeneboronic acid, (3-cyanomethylphenyl)boronic acid, and derivatives thereof, including boronic esters formed by reaction of boronic acid with an alcohol. Examples of boronic esters include, but are not limited to, allylboronic acid pinacol ester, phenyl boronic acid trimethylene glycol ester, diisopropoxy-methylborane, bis(hexyleneglycolato)diboron, t-butyl-N-[4-(4,4,5,5-tetramethyl-1,3,2-dioxaborolan-2-yl)phenyl]carbamate, 2,6-dimethyl-4-(4,4,5,5-tetramethyl-1,3,2-dioxaborolan-2-yl)benzoate, 4-(4,4,5,5-tetramethyl-1,3,2-dioxaborolan-2-yl)aniline, 4-(4,4,5,5-tetramethyl-1,3,2-dioxaborolan-2-yl)benzoic acid, 4-(4,4,5,5-tetramethyl 1,3,2-dioxaborolan-2-yl)phenol, 2-methoxy-4-(4,4,5,5-tetramethyl-1,3,2-dioxaborolan-2-yl)phenol, and the like.

In some embodiments, the formulation further comprises one or more excipients including, but not limited to, glyceryl monooleate, lecithin, oleic acid, dibutyl sebacate salts, such as NaCl, preservatives, glycerin, chlorhexidine, dimethyl sulfoxide, glyceryl behenate, Na stearate, glyceryl palmitostearate, olive oil, and sucrose stearate.

In particular embodiments, the degradable polymer is poly(lactic-coglycolic acid) (PLGA). Accordingly, in some embodiments, the presently disclosed formulation is a chitosan-coated, poly(lactic-coglycolic acid) (PLGA)-based sustained-release mucoadhesive formulation.

In certain aspects, the mucoadhesive formulation further comprises one or more surfactants. In particular aspects, the one or more surfactants are selected from the group consisting of polyoxyethylene sorbitol ester and sorbitan oleate.

As used herein, a “mucosal surface” is a surface that is lined by epithelial cells that form a physical barrier protecting the body against external noxious substances and pathogens. More particularly, a “mucous membrane” or “mucosa” is a membrane that lines various cavities in the body and covers the surface of internal organs. The mucosa consists of one or more layers of epithelial cells overlying a layer of loose connective tissue and is continuous with the skin at various body openings including, but not limited to, the eyes, ears, nose, mouth, lip, vagina, the urethral opening, and the anus. Particular mucosa include bronchial mucosa and the lining of vocal folds; endometrium (the mucosa of the uterus); esophageal mucosa; gastric mucosa; intestinal mucosa; nasal mucosa; olfactory mucosa; oral mucosa; ocular mucosa, endometrium, penile mucosa; vaginal mucosa; frenulum of tongue; tongue; anal canal; and palpebral conjunctiva.

As used herein, a “mucoadhesive” compound refers to a compound that adheres to a mucosal surface or submucosa. More particularly, a mucoadhesive compound will generally recognize and bind to a constituent of the target mucosal surface, including a glycoprotein, such as a mucin, a receptor, a polysaccharide, or other constituent. In so doing, mucoadhesive compounds increase the amount of time the mucoadhesive compound, or therapeutic agent associated therewith, is in contact with a mucosal surface.

Representative mucoadhesive compounds include chitosan, chitosan salts, or chitosan derivatives, such as thiolated chitosans. Representative chitosan salts include, but are not limited to, chitosan acetate, chitosan lactate, chitosan formate, chitosan maleate, chitosan chloride, chitosan ascorbate, chitosan citrate, and combinations thereof. One of ordinary skill in the art would recognize that other mucoadhesive compounds are suitable for use with the presently disclosed formulations.

This formulation can be used to control GI bleeding from cancers that cause surface-based oozing type bleeding and to cover the dissection site after removal of diseased tissue, such as in polyp removal (FIG. 11). This mucoadhesive polymer is expected to have high adhesive affinity to the sub-mucosal fiber layer thereby reducing risk of immediate washing off. In some embodiments, the mucoadhesive formulation has a low viscosity. For example, the mucoadhesive formulation can have a viscosity ranging from about 0.01 cp to 3000 cp, including 0.01, 0.1, 1, 5, 10, 50, 100, 200, 300, 400, 500, 600, 700, 800, 900, 1000, 1500, 2000, 2,500, and 3000 cp. In some embodiments, the viscosity has a range from about 10 to about 100 cp, including 10, 20, 30, 40, 50, 60, 70, 80, 90, and 100 cp. In yet other embodiments, the viscosity has a range from about 8 to about 10 cp, including 8.0, 8.5, 9.0, 9.5, and 10 cp. In other embodiments, the mucoadhesive formulation has a high concentration of epinephrine. In yet other embodiments, the mucoadhesive formulation has a fast release of epinephrine.

A mucoadhesive epinephrine gel that can adhere to the GI mucosa has several advantages, such as prolonged residence at the site of application and better contact with the underlying mucosa, which increases bioavailability of epinephrine. The duration for which this gel can adhere to the GI mucosa is limited by the turnover of the mucus layers.

Many of these polymers have been previously used in FDA-approved mucoadhesive gel systems. The choice of polymer will influence the diffusion and rate of drug release, which can be variably controlled for different applications. Epinephrine being a drug with low molecular weight, the polymer in unlikely to cause diffusional hindrance.

This mucoadhesive polymer is expected to have high adhesive affinity to the sub-mucosal fiber layer thereby reducing risk of immediate washing off

In yet other embodiments, many endoscopic interventions involve use of both formulations. In such embodiments, the presently disclosed injectable formulation would be applied first, for example, injected into a depot to facilitate polyp removal, followed by application of a presently disclosed mucoadhesive formulation to control or prevent bleeding after polyp removal.

### I.A.3. Gel Formulations for the Sustained Release of Epinephrine Further Comprising One or More Local Anesthetics

In further embodiments, the presently disclosed epinephrine gels can be used in combination with one or more additional therapeutic agents. In particular embodiments, the one or more additional therapeutic agents is a local anesthetic. Such embodiments can be used for endoscopic interventions with prolonged duration to reduce intestinal spasm and improve visualization during endoscopy.

Representative local anesthetics suitable for use with the presently disclosed formulations include, but are not limited to, amino esters, such as benzocaine, chloroprocaine, cyclomethycaine, dimethocaine (larocaine), piperocaine, propoxycaine, procaine (novocaine), proparacaine, tetracaine (amethocaine); amino amides, such as articaine, bupivacaine, cinchocaine (dibucaine), etidocaine, levobupivacaine, lidocaine (lignocaine), mepivacaine, prilocaine, ropivacaine, trimecaine; and naturally derived local anesthetics, such as, saxitoxin, neosaxitoxin, tetrodotoxin, menthol, eugenol, cocaine, and spilanthol.

In certain embodiments, the local anesthetic is selected from the group consisting of prilocaine, lidocaine, bupivacaine, articaine, and combinations thereof. In more certain embodiments, the local anesthetic is a combination of one or more of: epinephrine and prilocaine; epinephrine, lidocaine, and bupivacaine; epinephrine and lidocaine (i.e., iontocaine), epinephrine and articaine (i.e., septocaine), and combinations thereof. In particular embodiments, the local anesthetic is lidocaine.

### I.B. Method for Preventing or Controlling a Gastrointestinal Bleed

In some embodiments, the presently disclosed subject matter provides a method for preventing or controlling a gastrointestinal bleed, the method comprising administering to a subject in need of treatment thereof a formulation comprising at least one of a presently disclosed injectable formulation, a presently disclosed mucoadhesive formulation, or combinations thereof.

In some embodiments, the gastrointestinal bleed is associated with a deep source of gastrointestinal bleeding. In particular embodiments, the deep source of gastrointestinal bleeding is associated with an ulcer. In yet more particular embodiments, the formulation comprises the presently disclosed injectable formulation.

In other embodiments, the gastrointestinal bleed is associated with a superficial source of gastrointestinal bleeding. In particular embodiments, the superficial source of gastrointestinal bleeding is associated with a cancerous lesion. In yet more particular embodiments, the formulation comprises the presently disclosed mucoadhesive formulation.

I.C. Method for Separating Diseased Tissue from Normal Tissue

In some embodiments, the presently disclosed subject matter provides a method for separating diseased tissue from normal tissue, the method comprising: (a) injecting an aliquot of the presently disclosed injectable formulation under the diseased tissue to form a depot thereunder, thereby lifting the diseased tissue from the normal tissue; and (b) dissecting the diseased tissue to separate the diseased tissue from the normal tissue at a dissection site. In certain embodiments, the diseased tissue comprises a polyp.

In certain embodiments, the method further comprises an endoscopic procedure selected from the group consisting of endoscopic mucosal resection (EMR), endoscopic sub mucosal dissection (ESD), endoscopic myotomy, third-space endoscopy, endoscopic tunneling, and combinations thereof.

In more certain embodiments, the method further comprises administering a presently disclosed mucoadhesive formulation to the dissection site to prevent or control bleeding thereof. In other embodiments, the presently disclosed subject matter provides a method for sealing a perforation in tissue of a GI tract, e.g., stomach tissue or intestinal tissue.

### I.D. Endoscopic Injection Needles

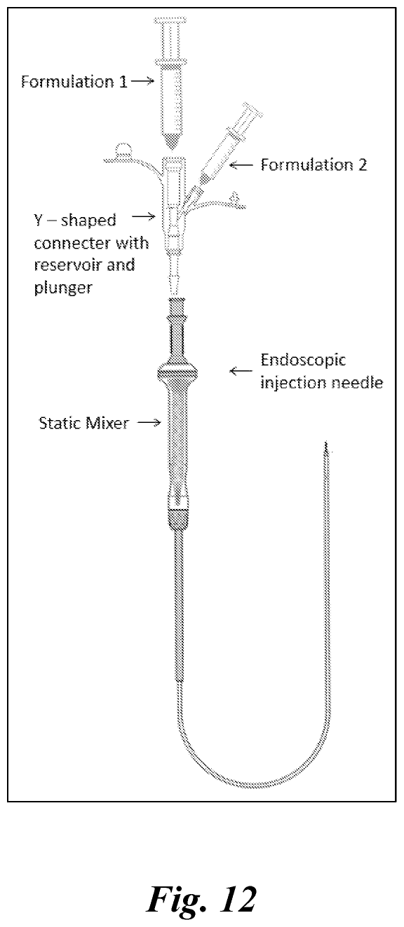

Referring now to FIG. 12, in some embodiments, the presently disclosed subject matter provides an endoscopic injection needle for delivering an injectable solution comprising a mixture of at least two formulations, e.g., in some embodiments, a presently disclosed injectable formulation and a presently disclosed mucoadhesive formulation, to customize the properties of formulations with regards to viscosity, epinephrine release rate and duration, to a tissue treatment site, the endoscopic injection needle comprising:

(a) a connecter comprising a proximal portion and a distal portion: (i) at least two inlet ports at the proximal portion of the connecter, wherein the at least two inlet ports are in fluid communication with a reservoir; (ii) an outlet port at the distal portion of the connector, wherein the outlet port is in fluid communication with the reservoir; and (iii) a plunger movably positionable within the proximal portion of the reservoir, the plunger providing a seal at the proximal portion of the connector to prevent the injectable solution from flowing out of the proximal portion of the connector and wherein the plunger further comprises a plunger advancing member configured to force the injectable solution from the reservoir through the outlet port at the distal portion of the connector;

(b) a static mixing chamber comprising a proximal portion and a distal portion, wherein the proximal portion of the static mixing chamber is in fluid communication with the outlet port at the distal portion of the reservoir, wherein the static mixing chamber is configured to receive the injectable solution from the reservoir; and

(c) a sheath comprising a proximal portion and a distal portion, wherein the proximal portion of the sheath is in fluid communication with the distal portion of the static mixing chamber, and wherein the sheath further comprises a needle enclosed therein, wherein the distal portion of the sheath is movable to expose the needle for insertion into the tissue treatment site. One of ordinary skill in the art would recognize that the needle can be a needle, cannula or other elongate tubular structure suitable for insertion into the tissue treatment site.

In operation, the needle is inserted between a first layer of tissue and a second layer of tissue. Injection of, for example, a presently disclosed injectable formulation, forms a fluid-filled pocket, e.g., a depot, that forces separation between the first and second layers of tissue. The elevated tissue portion, e.g., diseased tissue can then be dissected by a physician using an electrocautery device or other dissection device known in the art.

As used herein, the terms “proximal” and “distal” should be understood as being in terms of a physician delivering the presently disclosed formulations to a patient. Accordingly, the term “distal” refers to the portion of the endoscopic needle, or a component thereof, that is farthest from the physician and the term “proximal” refers to the portion of the endoscopic needle, or component thereof, that is nearest to the physician.

As used herein, the term “static mixing chamber” includes any static mixer known in the art designed for the continuous mixing of fluid materials without moving components. In such components, the energy needed for mixing comes from a loss in pressure as fluids flow through the static mixer. One design of static mixer consists of mixer elements contained in a cylindrical or squared housing. In such designs, the static mixer elements consist of a series of baffles, which blend two streams of fluids as they move through the baffles. Typical materials for static mixer components included stainless steel, polypropylene, Teflon, PVDF, PVC, CPVC, polyacetal, and glass-lined steel.

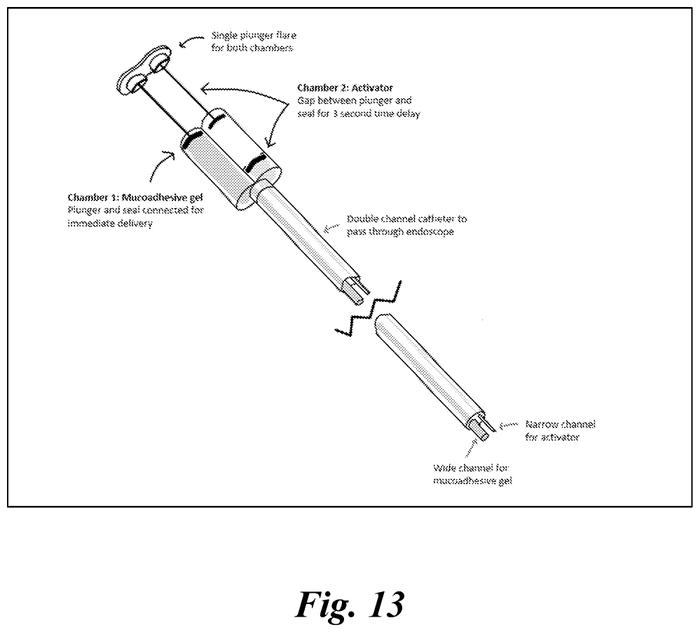

Referring now to FIG. 13, in other embodiments, the presently disclosed subject matter provides an endoscopic injection needle for delivering an injectable mucoadhesive gel formulation to a tissue treatment site, the endoscopic injection needle comprising:

(a) at least two inlet ports, wherein a first inlet port is in fluid communication with a first chamber for accommodating a mucoadhesive gel and a second inlet port is in fluid communication with a second chamber for accommodating an activator;

(b) at least two outlet channels, wherein a first outlet channel is in fluid communication with the first chamber and a second outlet channel is in fluid communication with the second chamber; and

(c) a first plunger and a second plunger movably positionable within a proximal portion of the first chamber and a proximal portion of the second chamber, the first and second plunger providing a seal at the proximal portion of the first and second chamber to prevent the mucoadhesive gel injectable solution from flowing out of the proximal portion of the first chamber and the activator from flowing out of the proximal portion of the second chamber, wherein the first plunger and the second plunger further comprise a single plunger advancing member configured to force the mucoadhesive gel from the first chamber through the first outlet channel and the activator from the second chamber through the second outlet channel,

wherein the plunger and seal of the second chamber are operationally positioned to form a gap to delay delivery of the activator in relation to delivery of the mucoadhesive gel to the tissue treatment site.

In some embodiments, the first outlet channel for dispensing the mucoadhesive gel has a larger diameter, i.e., is wider, than the second outlet channel for dispensing the activator.

### I.E. Kits

In some embodiments, the presently disclosed subject matter provides a kit comprising at least one of a presently disclosed injectable formulation, a presently disclosed mucoadhesive formulation, or combinations thereof. In certain embodiments, the kit further comprises an endoscopic injection needle as disclosed herein. In particular embodiments, the kit further comprises instructions for use.

### I.F. Methods for Delivering One or More Therapeutic Agents to a Targeted Site

In other embodiments, the presently disclosed subject matter provides a method for delivering one or more therapeutic agents to a targeted site in a gastrointestinal (GI) tract, the method comprising administering a presently disclosed formulation with endoscopy to the targeted site.

In such embodiments, the presently disclosed formulations can form a drug depot or reservoir in the sub-mucosa of the GI tract, which is created after tunneling in sub-mucosa for the sustained release of drugs and therapeutic agents.

In certain embodiments, the one or more therapeutic agents are selected from the group consisting of one or more corticosteroids, including, but not limited to, triamcinolone, budesonide, prednisone, and the like, one or more antibiotics, one or more chemotherapeutic agents, such as Mitomycin-C, one or more tumor necrosis factor inhibitors, such as Etanercept, one or more angiogenesis inhibitors, such as Thalidomide, one or more kinase inhibitors, such as Imatinib, one or more immunosuppressive agents, including, but not limited to, Tacrolimus, Sirolimus, and Azathioprine, one or more 5-aminosalicylic acid (5-ASA) agents, including, but not limited to, mesalamine, balsalazide, olsalazine, and sulfasalazine, polytetrafluoroethylene, one or more silicone-based gels, polyacrylamide, polyacrylonitrile, and combinations thereof.

In certain embodiments, the presently disclosed method further comprises treating or preventing one or more diseases, disorders, or conditions selected from the group consisting of one or more strictures in an esophagus or intestine, one or more infected collections around a GI tract, dysmotility or incontinence, inflammatory bowel disease (IBD) and related inflammation, one or more fistulae, and inflammation in liver, pancreas, stomach, intestine, and combinations thereof.

### II. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### In Vitro Evaluation Injectable Gel

For the injectable gel, representative polymers of interest, including Chitosan, triblock PEO-PPO-PEO block copolymers of poly(ethylene oxide) (PEO) and poly(propylene oxide) (PPO), Poly(D,L-lactide) (PDLA), Poly(D,L-lactide-co-glycolide) (PLGA), Oxyethylene Oxypropylene Polymer (Methyl Oxirane polymer with Oxirane), Oxyethylene Oxypropylene Polymer, Polyvinylpyrrolidone were evaluated with in-vitro experiments including a viscosity assay and release/permeation tests.

1.1 Viscosity assay. Rheological characteristics were evaluated at 25° C. and 37° C. using the cone and plate method with the HR-2 Discovery Hybrid Rheometer. Each experimental run consists of an “upcurve” where stress is measured as rotational speed is increased, followed by a “downcurve” where stress is measured as rotational speed is ramped down. Duplicate runs were performed for each item at 25° C. to verify reproducibility, and an additional run was performed at 37° C. to evaluate the effect of increased temperature. The rheological characteristics were assessed at 10 Pascal (Pa) and 50 Pa shear stress to mimic the pressures within gastrointestinal tissue and an endoscopy injection needle respectively. Referring now to Table 1, polymers of interest of different concentrations were combined with epinephrine and pigment to evaluate their viscosities and the feasibility of being able to inject them using conventionally available endoscopic injection accessories. The upper limit of polymer concentrations was identified with this assay.

FIG. 3 are graphs showing the viscosity as a function of temperature and shear rate vs shear stress. The first two graphs (FIG. 3A and FIG. 3B) showed the viscosity as a function of temperature, and these graphs show how the viscosity changes as a function of temperature, this is important to show that the gel product undergoes reverse thermal gelation for some of the polymers as the temperature increases the viscosity increases. The behavior helps the polymer to stay in place and not dissipate when injected into the tissue. This is an advantageous property as the submucosal injection list (FIG. 5) formation facilities the surgical procedure.

The remaining graphs in FIG. 3C, FIG. 3D, FIG. 3D, FIG. 3E and FIG. 3F show the rheological properties of the polymers as a function of shear rate and shear stress. For these graphs, the curvature indicates non-Newtonian behavior. These gels show shear thinning. Thus, when being injected and going through the tubing of the injection needle (FIG. 6A and FIG. 6B) the viscosity decreases, and when at rest it increases again, giving the submucosal lift a better chance of staying in place while reducing the amount of force or difficulty in injecting the gel that is to form in the tissue. Some of the graphs show hysteresis, this means that as the shear rate is increasing the viscosity (related to the slope of the line) is different from when the shear rate is decreasing, the up vs down viscosity. This gives an indication of how fast it takes the viscosity to change after undergoing shear thinning.

The most suitable polymers for injection were identified with these assays.

TABLE 1A
In-vitro experiment evaluating viscosity of gel formulations at
room (25° C.) and body (37° C.) temperature at a high-
stress environment mimicking pressure in the endoscopy injection needle

| | | Apparent Viscosity | |
|---|---|---|---|
| | | at 50 Pa Shear | |
| | | Stress (Pa · s) | |
| Formulation (% w/v) | Acronym | 25° C. | 37° C. |
| 10% triblock PEO-PPO-PEO copolymers of | PF10 | 8.60 × 10⁻³ | 1.24 × 10⁻² |
| poly(ethylene oxide) (PEO) and poly(propylene | | | |
| oxide) (PPO) | | | |
| 11% triblock PEO-PPO-PEO copolymers of | PF11 | 1.06 × 10⁻² | 1.12 × 10⁻² |
| poly(ethylene oxide) (PEO) and poly(propylene | | | |
| oxide) (PPO) | | | |
| 12% triblock PEO-PPO-PEO copolymers of | PF12 | 1.49 × 10⁻² | 1.85 × 10⁻² |
| poly(ethylene oxide) (PEO) and poly(propylene | | | |
| oxide) (PPO) | | | |
| 15% triblock PEO-PPO-PEO copolymers of | PF15 | 3.05 × 10⁻² | 3.86 × 10⁻² |
| poly(ethylene oxide) (PEO) and poly(propylene | | | |
| oxide) (PPO) | | | |
| 17% triblock PEO-PPO-PEO copolymers of | PF17 | 8.58 × 10⁻² | 3.37 |
| poly(ethylene oxide) (PEO) and poly(propylene | | | |
| oxide) (PPO) | | | |
| 0.8% Chitosan (L) | CL0.8 | 1.15 × 10⁻² | 8.03 × 10⁻³ |
| 1% Chitosan (L) | CL1 | 4.70 × 10⁻² | 3.64 × 10⁻² |
| 1% Chitosan (H) | CH1 | 3.58 × 10⁻¹ | 2.33 × 10⁻¹ |
| 1% Chitosan (S) | CHS | 3.87 × 10⁻¹ | 1.22 × 10⁻¹ |
| 2% Chitosan (L) | CL2 | 2.81 × 10⁻¹ | 1.85 × 10⁻¹ |
| 5% Chitosan (L) | CL5 | 5.75 | 4.26 |
| 2% Xanthan Gum | XG2 | 9.94 × 10⁻¹ | 9.91 × 10⁻¹ |
| 13% Methyl Oxirane polymer with Oxirane | KP338G13 | 1.76 × 10⁻² | 5.05 × 10⁻² |
| 15% Methyl Oxirane polymer with Oxirane | KP338G15 | 2.99 × 10⁻² | 9.95 × 10⁻² |
| 17% Methyl Oxirane polymer with Oxirane | KP338G17 | 4.46 × 10⁻² | 3.17 |
| 10% Polyvinylpyrrolidone | K90F10 | 2.38 × 10⁻¹ | 1.78 × 10⁻¹ |
| 17% Methyl Oxirane polymer with Oxirane:5% | G17 + CL5 | 8.28 × 10⁻² | 8.67 × 10⁻² |
| Chitosan (L) = 17:3 | | | |

‡S, L, H differ in molecular weights

TABLE 1B
In-vitro experiment evaluating viscosity of gel formulations
at room (25° C.) and body (37° C.) temperature at
a low-stress environment mimicking pressure in the gastrointestinal tissue

| | | Apparent Viscosity | |
|---|---|---|---|
| | | at 10 Pa Shear | |
| | | Stress (Pa · s) | |
| Formulation (% w/v) | Acronym | 25° C. | 37° C. |
| 1% triblock PEO-PPO-PEO copolymers of | PF1 | 1.52 × 10⁻³ | 1.12 × 10⁻³ |
| poly(ethylene oxide) (PEO) and poly(propylene | | | |
| oxide) (PPO) | | | |
| 2% triblock PEO-PPO-PEO copolymers of | PF2 | 1.92 × 10⁻³ | 1.52 × 10⁻³ |
| poly(ethylene oxide) (PEO) and poly(propylene | | | |
| oxide) (PPO) | | | |
| 5% triblock PEO-PPO-PEO copolymers of | PF5 | 3.31 × 10⁻³ | 3.72 × 10⁻³ |
| poly(ethylene oxide) (PEO) and poly(propylene | | | |
| oxide) (PPO) | | | |
| 10% triblock PEO-PPO-PEO copolymers of | PF10 | 7.92 × 10⁻³ | 8.70 × 10⁻³ |
| poly(ethylene oxide) (PEO) and poly(propylene | | | |
| oxide) (PPO) | | | |
| 11% triblock PEO-PPO-PEO copolymers of | PF 11 | 9.43 × 10⁻³ | 9.58 × 10⁻³ |
| poly(ethylene oxide) (PEO) and poly(propylene | | | |
| oxide) (PPO) | | | |
| 12% triblock PEO-PPO-PEO copolymers of | PF12 | 1.40 × 10⁻² | 1.62 × 10⁻² |
| poly(ethylene oxide) (PEO) and poly(propylene | | | |
| oxide) (PPO) | | | |
| 13% triblock PEO-PPO-PEO copolymers of | PF13 | 1.86 × 10⁻² | 2.93 × 10⁻² |
| poly(ethylene oxide) (PEO) and poly(propylene | | | |
| oxide) (PPO) | | | |
| 15% triblock PEO-PPO-PEO copolymers of | PF15 | 3.17 × 10⁻² | 4.2 × 10⁻² |
| poly(ethylene oxide) (PEO) and poly(propylene | | | |
| oxide) (PPO) | | | |
| 0.8% Chitosan (L) | CL0.8 | 1.54 × 10⁻² | 9.59 × 10⁻³ |
| 1% Chitosan (L) | CL1 | 7.84 × 10⁻² | 5.09 × 10⁻² |
| 1% Oxyethylene Oxypropylene Polymer | KP118G1 | 1.03 × 10⁻³ | Low of range |
| 10% Oxyethylene Oxypropylene Polymer | KP118G10 | 4.51 × 10⁻³ | 4.31 × 10⁻³ |
| 1% Methyl Oxirane polymer with Oxirane | KP338G1 | 1.55 × 10⁻³ | Low of range |
| 10% Methyl Oxirane polymer with Oxirane | KP338G10 | 1.45 × 10⁻² | 1.90 × 10⁻² |
| 13% Methyl Oxirane polymer with Oxirane | KP338G13 | 1.75 × 10⁻² | 5.62 × 10⁻² |
| 15% Methyl Oxirane polymer with Oxirane | KP338G15 | 2.92 × 10⁻² | N/A |
| 1% Oxyethylene Oxypropylene Polymer | KP407G1 | 9.62 × 10⁻⁴ | Low of range |
| 10% Oxyethylene Oxypropylene Polymer | KP407G10 | 7.52 × 10⁻³ | 7.89 × 10⁻³ |
| 1% Polyvinylpyrrolidone (L) | K90F1 | 3.49 × 10⁻³ | Low of range |
| 1% Polyvinylpyrrolidone (H) | K30O1 | 1.54 × 10⁻³ | Low of range |
| 10% Polyvinylpyrrolidone (H) | K30O10 | Low of range | 4.59 × 10⁻³ |
| 15% Polyvinylpyrrolidone (H) | K30O15 | 1.10 × 10⁻² | 8.21 × 10⁻³ |

‡S, L, H differ in molecular weights

1.2 Release/permeation tests. In Vitro epinephrine release study was performed using vertical diffusion cell system (FIG. 4A) (MIXdrive 6, Hanson Research Corporation). Before testing samples, diffusion cell volume needs to be determined. 10 mL syringe was filled with distilled water, and the weight of the filled syringe was determined. One of the cell was filled with distilled water using the syringe. Then the syringe was removed and weighed. So the weight of water required to fill the cell was determined, and the cell volume 6.78 mL is obtained (assuming the density of the water is 1 g/mL). Water bath circulator was set up to 37° C. PBS 1× (Phosphate Buffered Saline 1×) was used as the replacement media and pumped into the cell to form a positive meniscus at the top of the cell. A membrane (molecular cut off is 14,000) was place on top of one cell in contact with the positive meniscus of the media. A donor cell disc was put and secured on top of the membrane to hold the sample. Gels with and without epinephrine were then taken with 1 mL syringe. The syringe with gel was weighed and recorded as gross weight (Gross Wt.). After adding the sample to one donor cell disc of the diffusion system, the syringe was weighed and recorded as tare weight (Tare Wt.). Then sample weight added to the cell was obtained and recorded as net weight (Net Wt.) by using the equation Net Wt.=Gross Wt.−Tare Wt. According to the concentration of epinephrine in the gel, total amount of epinephrine in the sample can be calculated and recorded as theoretical epinephrine amount (Theoretical Epinephrine Amt.) by using the equation Theoretical Epinephrine Amt.=Net Wt.*Epinephrine Conc. This was the endpoint of the epinephrine release study. A standard curve of epinephrine was obtained and used to calculate epinephrine concentrations of samples (Sample Conc.).

After 0.5 h, 0.75 h, 1 h, 1.5 h, 2 h, 4 h, 24 h, 48 h, and 72 h, a sample was taken out and the same amount of replacement media was pumped into the diffusion cell. Samples were diluted 5 fold and tested using UV-Vis instrument at 280 nm. FIG. 4B, FIG. 4C shows that epinephrine can permeate across the mucosa for local effect. This experiment demonstrates that epinephrine can release for 72 hours. The diffusion capacity and release rate can be modified by changing the polymer type or viscosity and epinephrine concentration.

TABLE 2
Representative gel formulations examined
in-vitro to determine the release of epinephrine
over time in a vertical diffusion system (FIG. 4A)

| | | | | Epi- | | |
|---|---|---|---|---|---|---|
| | | | Conc. | nephrine | | |
| | | | (% | Conc. | CaCl₂ | CaCl₂ |
| No. | Acronym | Ingredients | w/v) | (mg/ml) | Conc. | Amt. |
| 1 | Alg1.5E10 | Sodium Alginate | 1.5 | 0.0796 | 0 | 0 |
| 2 | Alg1.5E10+1MCaCl₂ | Sodium Alginate, | 1.5 | 0.0796 | 1M | 0.5 ml |
| | | Calcium Chloride | | | | |
| 3 | APT20E10 | Sodium Alginate, | | 0.0796 | 0 | 0 |
| | | PEO, polyoxy- | | | | |
| | | ethylene sorbitol | | | | |
| | | ester | | | | |
| 4 | APT20E10+1MCaCl₂ | Sodium Alginate, | | 0.0796 | 1M | 0.5 ml |
| | | PEO, polyoxy- | | | | |
| | | ethylene sorbitol | | | | |
| | | ester, Calcium | | | | |
| | | Chloride | | | | |
| 5 | KPE10 | Methyl Oxirane | 6.5/ | 0.0796 | 0 | 0 |
| | Inject. | polymer with | 0.6 | | | |
| | | Oxirane, PEO | | | | |

### Example 2

### In Vitro Evaluation Injectable Gel with Epinephrine Nanoparticles

In Vitro epinephrine release study in the EXAMPLE 1.2 was repeated using epinephrine nanoparticles to allow a sustained release over at least 72 hours. (FIG. 4C). We revised the procedure of Wang et al. to make the particles. PEG-PLGA is dissolved in organic solvent (acetonitrile:methanol=1:1 by volume), then an aliquot of methanol was taken from the drug solution and mixed with a dissolved polymer. Organic mixture was then sonicated until no particle could be seen. Then, 0.5% polyoxyethylene sorbitan monooleate solution is added dropwise into organic solvent mixture with polymer, which is then sonicated for 0.5 min, which forms an emulsion. The emulsion is added to 0.5% polyoxyethylene sorbitan monooleate solution, which is stirred overnight to let organic solvent volatilize. Then nanoparticles are washed thrice to remove surfactant with deionized water using 13,000 g centrifugation at 4° C. Particle size and zeta potential of these prepared placebo nanoparticles are measured using Zetasizer. The particle size of epinephrine bitartrate nanoparticles is 113.4+/−0.55 nm, PDI (polydispersity index) is 0.109+/−0.021. Zeta potential is −24.6+/−0.36 mV. FIG. 6 demonstrates that representative gel formulations with epinephrine nanoparticles greatly extended the duration of release of epinephrine to at least 72 hours.

This study shows the feasibility of using sustained release epinephrine in an injectable formulation and mucoadhesive gels using a combination of degradable gel polymers, free epinephrine and epinephrine nanoparticles.

### Example 3

### Ex Vivo Evaluation Injectable Gel for the Feasibility of Injection

Porcine gastrointestinal tissue was used to evaluate the feasibility of injecting the gel polymers of interest using conventional endoscopy injection needles (FIG. 5A). This experiment confirmed that the depots having a volume of about 10 mL can be created with ease within the GI tissue. Depots of up to 40 mL were created within the porcine stomach tissue followed by dissection to demonstrate an intact depot.

### Example 4

### Ex Vivo Evaluation Injectable Gel Evaluating Submucosal Lift

In an extension to EXAMPLE 3, porcine gastrointestinal tissue was used to determine the duration for which the gel formulations when injected in the submucosal space will create adequate cushion lifting to facilitate endoscopic resection procedures (FIG. 5B). Porcine gastrointestinal tissue was fixed to the corkboard and gel formulations were injected into the submucosal space using conventional endoscopy injection needles. Using calipers the height of the submucosal cushion was measured for 45 minutes. The gastrointestinal specimens were maintained at 37° C. using temperature-controlled heating pads to mimic human body temperature. The duration of the submucoal lift was calculated using a cushion height decrease.

Cushion height decrease (%) was calculated as ((Height0−Height45)/Height0)×100 where Height0 and Height45 are the cushion height at t0=0 and after 45 minutes, respectively. The gel formulation with the optimum submucosal lift was determined through this study.

TABLE 3
Ex vivo experiment evaluating the adequacy of submucosal
tissue lift created by the injection gel formulations.
Smaller number is better performing

| | Cushion |
|---|---|
| | height |
| Formulation (% w/v) | decrease (%) |
| Hydroxyethyl starch (positive control) | 62.5 |
| Saline (negative control) | 83.5% |
| 1% triblock PEO-PPO-PEO copolymers of poly(ethylene | 50% |
| oxide) (PEO) and poly(propylene oxide) (PPO) | |
| 10% triblock PEO-PPO-PEO copolymers of poly(ethylene | 40% |
| oxide) (PEO) and poly(propylene oxide) (PPO) | |
| 0.8% Chitosan (L) | 87% |
| 12% triblock PEO-PPO-PEO copolymers of poly(ethylene | 35% |
| oxide) (PEO) and poly(propylene oxide) (PPO) | |
| 13% triblock PEO-PPO-PEO copolymers of poly(ethylene | 44% |
| oxide) (PEO) and poly(propylene oxide) (PPO) | |
| 1% Chitosan (H) | 42% |
| 10% Oxyethylene Oxypropylene Polymer | 36% |
| 13% triblock PEO-PPO-PEO copolymers of poly(ethylene | 20.5% |
| oxide) (PEO) and poly(propylene oxide) (PPO) | |
| 15% triblock PEO-PPO-PEO copolymers of poly(ethylene | 30% |
| oxide) (PEO) and poly(propylene oxide) (PPO) | |
| 1% Chitosan (L) | 29% |
| 13% triblock PEO-PPO-PEO copolymers of poly(ethylene | 70% |
| oxide) (PEO) and poly(propylene oxide) (PPO), | |
| 1% Chitosan (L) | |
| 15% triblock PEO-PPO-PEO copolymers of poly(ethylene | 52% |
| oxide) (PEO) and poly(propylene oxide) (PPO) | |
| Commercial gel (Orise ®) | 56% |
| Commercial gel (Everlift ®) | 60% |
| Commercial gel (Eleview ®) | 55% |

### Example 5

### Ex Vivo Evaluation of Injectable Gel for Injection Pressure

Porcine gastrointestinal tissue was used to evaluate the pressure of injecting the gel polymers using a conventional injection needle (23 G Olympus needle master). A pressure gauge was connected to the injection needle the average pressure to inject 5 mL of the gel formulation was determined. Positive control (hydroxyethyl starch), negative control (saline), and commercial gels were used for comparison. The feasibility of injection through endoscopy needle with ease was confirmed using this study.

TABLE 4
Ex vivo experiment evaluating the pressure of
injection through an endoscopy injection needle

| Formulation | Average Pressure (kPa) |
|---|---|
| Methyl Oxirane polymer with Oxirane | 133 |
| Methyl Oxirane polymer with Oxirane, PEO | 233 |
| Methyl Oxirane polymer with Oxirane, | 193 |
| epinephrine 1:100,000 | |
| Methyl Oxirane polymer with Oxirane, PEO, | 256 |
| epinephrine 1:100,000 | |
| Saline (negative control) | 58 |
| Hydroxyethyl starch (positive control) | 100 |
| Commercial gel (Orise ®) | 183 |
| Commercial gel (Everlift ®) | 158 |

### Example 6

### In Vivo Evaluation of Injectable Gel for Submucosal Lift

Representative gel polymers of interest were injected into a live pig stomach using an endoscope with a conventional injection needle (23 G Olympus needle master) (FIG. 6A, FIG. 6B and FIG. 6C). Depots of up to 40 mL were created in gastric submucosa. For polymers with a higher viscosity, a Boston Scientific Encore Inflator was attached to the injection needle to facilitate injection. In situ gelation was demonstrated in this experiment. Under direct endoscopic visualization, the presence or absence of a submucosal cushion was identified (Table 5). Cushion being present for at least 45 minutes was required for optimum performance and this was compared with positive and negative control, other commercially available lifting gel respectively.

After 72 hours of injection, the animal was sacrificed and an autopsy was performed. The depots at the site of polymer injection did not show any adverse effect on gross examination (FIG. 6D). On microscopic examination, minimal inflammation at the site of polymer injection was observed without any significant immune reaction or adverse effect, demonstrating safety of use of these polymers in GI tissue (FIG. 6E). Partial resorption of the gel depot also was observed.

TABLE 5
In vivo experiment evaluating the presence of submucosal
cushion for at least 45 minutes when injected
in liver porcine gastrointestinal tissue

| | Time at | Time at | Time at |
|---|---|---|---|
| | 15 min, | 30 min, | 45 min, |
| | cushion | cushion | cushion |
| Formulation | present | present | present |
| Methyl Oxirane polymer with Oxirane | Yes | Yes | Yes |
| Methyl Oxirane polymer with Oxirane, PEO | Yes | Yes | Yes |
| Methyl Oxirane polymer with | Yes | Yes | Yes |
| Oxirane, epinephrine 1:100,000 | | | |
| Methyl Oxirane polymer with Oxirane, | Yes | Yes | Yes |
| PEO, epinephrine 1:100,000 | | | |
| Saline (negative control) | Yes | No | No |
| Hydroxyethyl starch (positive | Yes | Yes | No |
| control) | | | |
| Commercial gel (Orise ®) | Yes | Yes | Yes |
| Commercial gel (Everlift ®) | Yes | Yes | No |

### Example 7

### In Vivo Evaluation of Injectable Gel for Bleeding Control

To test the efficacy of epinephrine gels for bleeding control, a porcine bleeding model was created. This was done by feeding 60-70 kg Yorkshire pigs with 2000 mg aspirin, 600 mg clopidogrel per day for 5 days. This was followed by injection of bolus low molecular weight heparin 50,000 units and 10,000 units per hour during endoscopy. Polyps were recreated in pig stomach and colon by submucosal injection of normal saline. Ulcers were recreated in the stomach and colon by using endoscopic submucosal dissection (ESD) and endoscopic mucosal resection (EMR) techniques described elsewhere. This recreated oozing ulcers in the pig stomach and colon.

After ulcer creation, gel formulations or saline (negative control), epinephrine 1:10,000 (positive control) of 5 mL each were injected adjacent to the bleeding ulcer. The bleeding ulcers were observed for 15 minutes each for the persistence or resolution of bleeding (Table 6). Methyl Oxirane polymer with Oxirane in combination with PEO, epinephrine nanoparticle, and 1:100,000 epinephrine could stop the bleeding from the ulcers and avoid recurrence. After 72 hours of injection, the animal was sacrificed and an autopsy was performed. The depots at the site of polymer injection did not show any adverse effect on gross examination. On microscopic examination, minimal inflammation at the site of polymer injection was observed without any significant immune reaction or adverse effect, demonstrating the safety of use and efficacy.

TABLE 6
In vivo experiment evaluating the efficacy of representative injectable
gels in treating bleeding ulcers in a live porcine model

| | Time at | Time at | Time at |
|---|---|---|---|
| | 5 min, | 10 min, | 15 min, |
| | bleeding | bleeding | bleeding |
| Formulation | present | present | present |
| Methyl Oxirane polymer with | No | Yes | Yes |
| Oxirane, PEO | | | |
| Methyl Oxirane polymer with | No | No | No |
| Oxirane, PEO, epinephrine | | | |
| 1:100,000, epinephrine | | | |
| nanoparticle | | | |
| Saline (negative control) | Yes | Yes | Yes |
| Epinephrine 1:10,000 | No | No | No |
| (positive control) | | | |

### Example 8

### In Vitro Evaluation of Mucoadhesive Gel

For the mucoadhesive gel, representative polymers of interest, including alginate (Alg) polyethylene oxide (PEO), methacrylic acid, and methyl methacrylate (E), Hydroxypropylcellulose (HPC), Carboxymethyl cellulose (CMC), and in combinations with surfactants polyoxyethylene sorbitol ester, Sorbitan oleate were evaluated with in-vitro experiments including a viscosity assay and release/permeation tests.

7.1 Mucoadhesion assay: The force of adhesion for the representative gel polymers was evaluated using an oscillation Frequency method at 37° C. in a Rheometer. The angular frequency was changed from 0.1 to 100.0 rad/s. Tan (delta) gave the adhesion parameters and a higher Tan (delta) means the gels tend to be more adhesive. This was calculated using the formula Tan (delta)=G″/G′=Loss Modulus/Storage Modulus, G″ relates to adhesion, G′ relates to cohesion. Complex Viscosity was calculated using the formula √((η′)\hat{ }2+(η″)\hat{ }2)=√((G\hat{ }′/ω))\hat{ }2±(G\hat{ }″/ω))\hat{ }2), ω is the angular frequency, complex viscosity relates to viscosity. Higher complex viscosity means a higher viscosity.

To improve the mucoadhesion using cross-linking of polymer, the gels were activated using divalent ions in the form of Ca2+, Mg2+, Ba2+, Sr2+, Pb2+, Cu2+, Cd2+, Zn2+, Ni2+ or Co2+.

TABLE 7
In-vitro experiment evaluating the mucoadhesive properties of gel formulations

| | | Tan | Complex |
|---|---|---|---|
| Acronym | Formulation (% w/v) | (delta)_max | Viscosity_max |
| Alg1 | 1% Sodium Alginate | 19.79 | 0.416 |
| Alg1.5 | 1.5% Sodium Alginate | 79.47 | 1.47 |
| Alg2.5 | 2.5% Sodium Alginate | 38.16 | 12.38 |
| PEO1 | 1% PEO | 3.67 | 2.18 |
| PEO2.5 | 2.5% PEO | 1.39 | 104.95 |
| APT20_1% | Alg2.5:PEO1 = 1:1 with 1% | 4.69 | 6.32 |
| | polyoxyethylene sorbitol ester | | |
| APT20_0.5% | Alg2.5:PEO1 = 1:1 with 0.5% | 5.87 | 6.05 |
| | polyoxyethylene sorbitol ester | | |
| APS80_0.5% | Alg2.5:PEO1 = 1:1 with 0.5% | 3.97 | 7.68 |
| | Sorbitan oleate | | |
| Alg2.5E1 | Alg2.5:methacrylic acid, and methyl | 21.71 | 0.89 |
| | methacrylate = 1:1 | | |
| Alg2.5E2 | Alg2.5:methacrylic acid, and methyl | 46.64 | 0.84 |
| | methacrylate = 1:1 | | |
| Alg2E2 | Alg2:methacrylic acid, and methyl | 17.7 | 1.54 |
| | methacrylate = 3:1 | | |
| Alg1.5E1 | 1.5% Sodium Alginate, 1% | 55.69 | 1.85 |
| | methacrylic acid, and methyl | | |
| | methacrylate | | |
| Alg 1.2 E 0.8 | 1.2% Sodium Alginate, 0.8% | 103.031 | 0.557 |
| | methacrylic acid, and methyl | | |
| | methacrylate | | |
| Alg1.5CMC0.1 | 1.5% Sodium Alginate, 0.1% Sodium | 19.65 | 3.31 |
| | Carboxymethyl cellulose | | |
| Alg1.5CMC0.5 | 1.5% Sodium Alginate, 0.5% Sodium | 9.1 | 7.23 |
| | Carboxymethyl cellulose | | |
| Alg1.5CMC1 | 1.5% Sodium Alginate, 1% Sodium | 4.53 | 20.45 |
| | Carboxymethyl cellulose | | |
| Alg1.5PEO2.5 | 1.5% Sodium Alginate, 2.5% PEO | 1.28 | 228 |
| HPC2 | 2% Hydroxypropylcellulose | | |
| HPC4 | 4% Hydroxypropylcellulose | 14.28 | 3.36 |

7.2 Release/permeation tests: Described in Example 1.2, and 2 this experiment demonstrates that epinephrine can release for at least 72 hours through the representative mucoadhesive gels containing epinephrine nanoparticles (FIG. 4C).

### Example 9

### Ex Vivo Evaluation of Mucoadhesive Gel for the Feasibility of Application

Porcine gastrointestinal tissue was used to evaluate the feasibility of applying the mucoadhesive gel to mucosal or mucosal tissue using hollow 7, 8.5, 10 Fr catheters. Submucosal exposure was created in porcine gastrointestinal tissue after removing the mucosal layer. After the gel application, Ca2+ was sprayed on the gel using a spray catheter. Ca2+ was delivered through CaCl₂ at 0.8, 0.9, 1, 1.2, 1.5, 2 M concentrations. The force of adhesion was assayed at 2, 3, 5, 10, 15, 20, 30 min (FIG. 8). The gels were exposed to HCl (pH 1.2) and a stream of saline to imitate the gastrointestinal environment. This experiment confirmed that the mucoadhesive gels can be applied to gastrointestinal tissue and activated using chemicals.

### Example 10

### In Vivo Evaluation of Mucoadhesive Gel for Bleeding Control

As described in example 7 porcine bleeding model was created (FIG. 9A) imitating ulcers from endoscopic dissection procedures including endoscopic mucosal resection (EMR) and endoscopic submucosal dissection (ESD). The representative adhesive gels were applied to the ulcer using 10 French catheters (FIG. 9B) followed by a spray of 0.8-1.2 M CaCl₂ activator on the gel surface (FIG. 9C). The ulcer bleeding was observed for 15 minutes and a firm adhesive layer was formed on the surface of the ulcer with no further bleeding noted (FIG. 9D). A water jet was applied using an endoscope on the adhered gel layer (FIG. 9E) and this was noted to be firmly adherent and did not wash off. This was repeated in the stomach and colon. After 24 hours, the animal was sacrificed and the adhesive gel layer was seen attached on the surface of the ulcer with no further bleeding (FIG. 9F). In other experiments, after 72 hours of injection, the animal was sacrificed and an autopsy was performed. The ulcer sites or adjacent gastrointestinal did not show any adverse effect on gross examination. On microscopic examination, minimal inflammation at the ulcer site was observed without any significant immune reaction or adverse effect, demonstrating the safety of use and efficacy.

TABLE 8
In vivo experiment evaluating the efficacy
of representative mucoadhesive gels in treating
bleeding ulcers in a live porcine model

| | | | |
|---|---|---|---|
| | Time at | Time at | Time at |
| | 5 min, | 10 min, | 15 min, |
| | bleeding | bleeding | bleeding |
| Formulation | present | present | present |

Stomach ulcers

| | | | |
|---|---|---|---|
| 1.5% w/v Sodium Alginate, 1M | No | No | No |
| CaCl₂, epinephrine- | | | |
| containing nanoparticle | | | |
| 1.2% w/v Sodium Alginate, | No | No | No |
| 0.8% w/v methacrylic acid, and | | | |
| methyl methacrylate, 1M CaCl₂, | | | |
| epinephrine-containing nanoparticle | | | |
| Saline (negative control) | Yes | Yes | Yes |

Colon ulcers

| | | | |
|---|---|---|---|
| 1.5% w/v Sodium Alginate, 1M | No | No | No |
| CaCl₂, epinephrine- | | | |
| containing nanoparticle | | | |
| 1.2% w/v Sodium Alginate, | No | No | No |
| 0.8% methacrylic acid, and | | | |
| methyl methacrylate, 1M CaCl₂, | | | |
| epinephrine-containing nanoparticle | | | |
| Saline (negative control) | Yes | Yes | Yes |

### Example 11

### Ex Vivo Evaluation of Mucoadhesive Gel for the Ability to Seal a Perforation

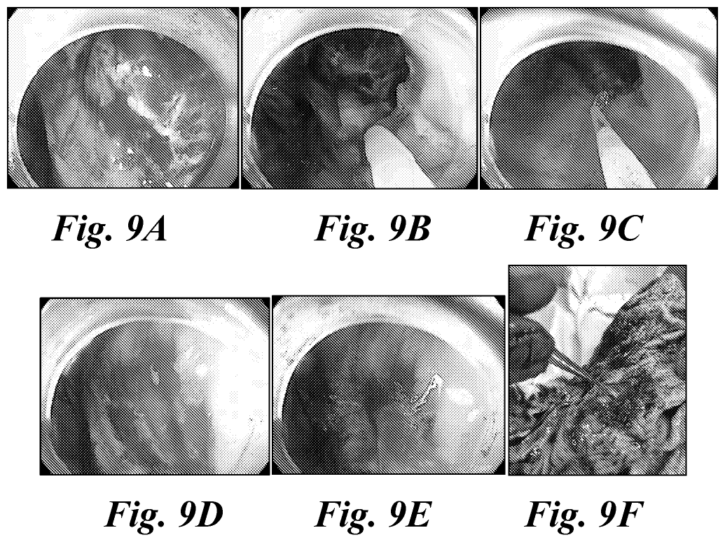

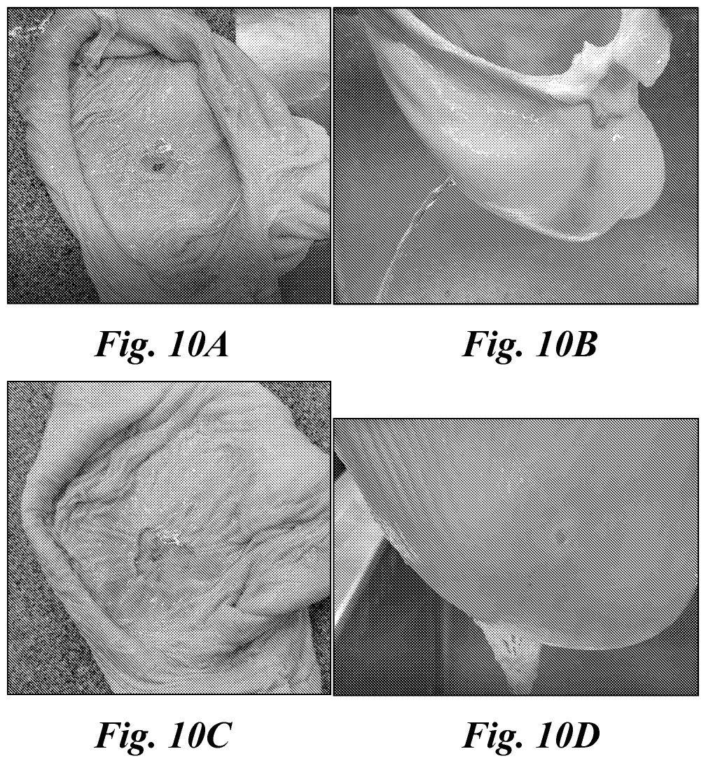

Gastrointestinal perforation may often occur during interventions including polypectomy, EMR, ESD. The efficacy of representative mucoadhesive gel was performed in porcine stomach tissue. A 3-mm perforation was created in the stomach (FIG. 10A). The pig stomach was filled with water and a clear leak could be identified (FIG. 10B). 1.2% w/v Sodium Alginate, 0.8% w/v methacrylic acid, and 0.8% w/v methyl methacrylate, 1 M CaCl₂ was then applied on the perforation defect and allowed to settle for 5 minutes (FIG. 10C). Water was filled into the stomach again, no further leak was identified at the perforation (FIG. 10D). This experiment demonstrates that the mucoadhesive gel can seal perforations in gastrointestinal tissue.

### Example 12

### Formulation 1: Injectable Gel

In representative embodiments of FORMULATION 1, an injectable gel, triblock PEO-PPO-PEO copolymers of poly(ethylene oxide) (PEO) and poly(propylene oxide) (PPO) can be used. Representative ingredients and amounts of the formulation are shown in the following table:

| Ingredient | Amount | |
|---|---|---|
| 13% triblock PEO-PPO-PEO | 6.5% | w/v |
| copolymers of poly(ethylene oxide) | | |
| (PEO) and poly(propylene oxide) (PPO) | | |
| Poly(ethylene oxide) (PEO) | 1.25% | w/v |
| Buffer | Na₂PO₄ | |
| | (anhydrous) 10 mM | |
| NaCl | 0.9% | w/v |
| Dye | 0.4 | mg |

| | |
|---|---|
| Ascorbic acid | Adjust pH to 6.5 |
| Sterile water for injection USP | Quantum satis (qs) |

Add gel polymers to a vessel with an overhead mixer, then add NaCl and Na₂PO₄ and mix until a clear solution is formed and then adjust pH to 6.5 with ascorbic acid and qs with water to final volume.

### Example 13

### Formulation 2: Injectable Gel with Epinephrine

In representative embodiments of FORMULATION 2, an injectable gel, triblock PEO-PPO-PEO copolymers of poly(ethylene oxide) (PEO) and poly(propylene oxide) (PPO) can be used in combination with nanoparticle coated epinephrine. Representative ingredients and amounts of the formulation are shown in the following table:

| Ingredient | Amount | |
|---|---|---|
| 13% triblock PEO-PPO-PEO | 6.5% | w/v |
| copolymers of poly(ethylene oxide) | | |
| (PEO) and poly(propylene oxide) (PPO) | | |
| Poly(ethylene oxide) (PEO) | 1.25% | w/v |
| Buffer | Na₂PO₄ | |
| | (anhydrous) 10 mM | |
| NaCl | 0.9% | w/v |
| Epinephrine immediate release | 0.08 | mg/mL |
| Epinephrine bitartrate nanoparticles | 0.04 | ml/mL |
| Dye | 0.4 | mg |

| | |
|---|---|
| Ascorbic acid | Adjust pH to 6.5 |
| Sterile water for injection USP | Quantum satis (qs) |

Using the production methods of EXAMPLE 13 once gel is made, add immediate release epinephrine and an epinephrine-containing nanoparticle to FORMULATION 1 using continuous stirring.

### Example 14

### Formulation 3: Mucoadhesive Gel

In representative embodiments of FORMULATION 3, a mucoadhesive gel, sodium alginate methacrylic acid, and methyl methacrylate can be used. This is activated using CaCl₂. Representative ingredients and amounts of the formulation are shown in the following table:

| Ingredient | Amount | |
|---|---|---|
| Sodium Alginate | 1.2% | w/v |
| Methacrylic acid, and methyl | 0.8% | w/v |
| methacrylate | | |
| Non-ionic detergent | 0.5% w/v polyoxyethylene | |
| | sorbitol ester | |
| Buffer | Na₂PO₄ | |
| | (anhydrous) 10 mM | |
| NaCl | 0.9% | w/v |
| Dye | 0.8 | mg |

| | |
|---|---|
| Ascorbic acid | Adjust pH to 6.5 |
| Sterile water for injection USP | Quantum satis (qs) |
| CaCl₂ | 1M |

Add polymers to a vessel and mix with an overhead mixer, then add NaCl and Na₂PO₄ and mix until a clear solution is formed and then adjust pH to 6.5 with ascorbic acid and qs with water to final volume.

### Example 15

### Formulation 4: Mucoadhesive Gel with Epinephrine

In representative embodiments of FORMULATION 4, a mucoadhesive gel, sodium alginate methacrylic acid, and methyl methacrylate can be used. This is activated using CaCl₂. Representative ingredients and amounts of the formulation are shown in the following table:

| Ingredient | Amount | |
|---|---|---|
| Sodium Alginate | 1.2% | w/v |
| Methacrylic acid, and methyl | 0.8% | w/v |
| methacrylate | | |
| Non-ionic detergent | 0.5% w/v polyoxyethylene | |
| | sorbitol ester | |
| Buffer | Na₂PO₄ | |
| | (anhydrous) 10 mM | |
| NaCl | 0.9% | w/v |
| Epinephrine immediate release | 0.08 | mg/mL |
| Epinephrine bitartrate nanoparticles | 0.04 | ml/mL |
| Dye | 0.8 | mg |

| | |
|---|---|
| Ascorbic acid | Adjust pH to 6.5 |
| Sterile water for injection USP | Quantum satis (qs) |
| CaCl₂ | 1M |

Using the production methods of EXAMPLE 14 once gel is made, add immediate release epinephrine and an epinephrine-containing nanoparticle to FORMULATION 3 using continuous stirring.

### Example 16

### Formulation 5: Mucoadhesive Gel with Epinephrine

In representative embodiments of FORMULATION 4, a mucoadhesive gel, sodium alginate methacrylic acid, and methyl methacrylate, PEO can be used. This is activated using CaCl₂. Representative ingredients and amounts of the formulation are shown in the following table:

| Ingredient | Amount | |
|---|---|---|
| Sodium Alginate | 1.2% | w/v |
| Methacrylic acid, and methyl | 0.8% | w/v |
| methacrylate | | |
| Poly(ethylene oxide) (PEO) | 0.5% | w/v |
| Non-ionic detergent | 0.5% w/v polyoxyethylene | |
| | sorbitol ester | |
| Buffer | Na₂PO₄ | |
| | (anhydrous) 10 mM | |
| NaCl | 0.9% | w/v |
| Epinephrine immediate release | 0.08 | mg/mL |
| Epinephrine bitartrate nanoparticles | 0.04 | ml/mL |
| Dye | 0.8 | mg |

| | |
|---|---|
| Ascorbic acid | Adjust pH to 6.5 |
| Sterile water for injection USP | Quantum satis (qs) |
| CaCl₂ | 1M |

Using the production methods of EXAMPLE 15 once gel is made, add immediate release epinephrine and an epinephrine-containing nanoparticle to FORMULATION 3 using continuous stirring.

### Example 17

### Photochemical Activated Mucoadhesive Gel (Prophetic)

One embodiment of this invention is the use of drug-eluting bioadhesive that has its mechanical properties enhanced via photochemical reactions. The PEO was modified using 3,4-Dihydroxyphenyl-L-alanine (DOPA) the PEO-DOPA was mixed with Sodium alginate. The mixture can be activated using UVA light and either 2-Hydroxy-4′-(2-hydroxyethoxy)-2-methylpropiophenone or 2-Hydroxy-1-(4-(2-hydroxyethoxy)phenyl)-2-methylpropan-1-one activator at 1%, which modifies the tissue binding. After photoactivation, the mixture can be further activated using CaCl₂. The advantage of this approach is that the combination of photochemical activation and ionic cross-linking creates strong bonds with the tissue, and has good mechanical properties, and the combination uses a smaller amount of activator, thus reducing the chance of tissue inflammation.

Other acrylates that can be used in combinations include but not limited to Polyethylene glycol in combination with acrylated poly-L lactid acid, trilyine amine, albumin, polyethyl amine; glutaraldehyde, polyaldehyde, cyanoacrylate, polyurethane, cyanoacrylate, dextran-urethanemethacrylate, sodium alginate conjugated either with 2-aminoethyl methacrylate, (AEMA), styryl-pyridine, or methacrylic anhydride; acrylated poly(glycerol sebacate) (PGS), poly(vinyl acetate) (PVA), PEG, and poly(ε-caprolactone) (PCL); acryloyl chloride (poly(glycerol sebacate acrylate) PGSA); PEG diacrylate (PEG-DA).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Gralnek I M, Barkun A N, Bardou M. Management of acute bleeding from a peptic ulcer. N Engl J Med 2008; 359:928-37.
Eisen G M, Baron T H, Dominitz J A, et al. Complications of upper GI endoscopy. Gastrointest Endosc 2002; 55:784-93.
Hwang J H, Fisher D A, Ben-Menachem T, et al. The role of endoscopy in the management of acute non-variceal upper GI bleeding. Gastrointest Endosc 2012; 75:1132-8.
Park C H, Lee S J, Park J H, et al. Optimal injection volume of epinephrine for endoscopic prevention of recurrent peptic ulcer bleeding. Gastrointest Endosc 2004; 60:875-80.
Sarmento Junior K MdA, Tomita S, Kós AOdÁ. Uso tópico da adrenalina em diferentes concentraçÕes na cirurgia endoscópica nasal. Revista Brasileira de Otorrinolaringologia 2009; 75:280-289.
Abel J, Crawford A. On the blood-pressure-raising constituent of the suprarenal capsule. Bull Johns Hopkins Hosp 1897; 8:151-7.
Akshintala V S, Hutfless S M, Colantuoni E, et al. Systematic review with network meta-analysis: pharmacological prophylaxis against post-ERCP pancreatitis. Aliment Pharmacol Ther 2013; 38:1325-37.
Kamal A, Akshintala V S, Talukdar R, et al. A Randomized Trial of Topical Epinephrine and Rectal Indomethacin for Preventing Post-Endoscopic Retrograde Cholangiopancreatography Pancreatitis in High-Risk Patients. Am J Gastroenterol 2019; 114:339-347.
Giday S A, Kim Y, Krishnamurty D M, et al. Long-term randomized controlled trial of a novel nanopowder hemostatic agent (TC-325) for control of severe arterial upper gastrointestinal bleeding in a porcine model. Endoscopy 2011; 43:296-9.
Peery A F, Dellon E S, Lund J, et al. Burden of gastrointestinal disease in the United States: 2012 update. Gastroenterology 2012; 143:1179-1187 e3.
Lee M F, Ma Z, Ananda A. A novel haemostatic agent based on self-assembling peptides in the setting of nasal endoscopic surgery, a case series. Int J Surg Case Rep 2017; 41:461-464.
Mohan B P, Asokkumar R, Yu J X, et al. Is Prophylactic Clipping Necessary After the Resection of Large (≥1 cm) Colorectal Lesions? If So, at What Cost?: 166. American Journal of Gastroenterology 2019; 114:S102.
Yu J X, Lin J L, Oliver M, et al. Trends in EMR for nonmalignant colorectal polyps in the United States. Gastrointest Endosc 2020; 91:124-131 e4.
https://www.biospace.com/article/releases/apollo-endosurgery-inc-secures-11-5-million-in-series-a-financing-/.
Sung J J, Luo D, Wu J C, et al. Early clinical experience of the safety and effectiveness of Hemospray in achieving hemostasis in patients with acute peptic ulcer bleeding. Endoscopy 2011; 43:291-5.
U.S. Pat. No. 10,611,908 for Micelles for mucoadhesive drug delivery to Sheardown et al., issued Apr. 7, 2020.
U.S. Pat. No. 10,568,836 for Epinephrine nanoparticles encapsulated with chitosan and tripolyphosphate, methods of fabrication thereof, and methods for use thereof for treatment of conditions responsive to epinephrine, to Rawas-Qalaji et al., issued Feb. 25, 2020.
U.S. Pat. No. 9,877,921 for Epinephrine nanoparticles, methods of fabrication thereof, and methods for use thereof for treatment of conditions responsive to epinephrine, to Rawas-Qalaji et al., issued Jan. 30, 2018.
U.S. Pat. No. 8,114,914 for Topical vasoconstrictor preparations and methods for protecting cells during cancer chemotherapy and radiotherapy, to Fahl et al., issued Feb. 14, 2012.
U.S. Pat. No. 6,319,260 for Method of endoscopic mucosal resection using mucopolysaccharide and local injection preparation, to Yamamoto, issued Nov. 20, 2001.
U.S. Patent Application Publication No. 20190350839 for Polyp lifting compositions and methods for use, to Edmondson et al., Nov. 21, 2019.
U.S. Patent Application Publication No. 20170189573 for Novel hemostatic patch and uses thereof, to Rubin et al., published Jul. 6, 2017.
U.S. Patent Application Publication No. 20150320694 for Mucoadhesive nanoparticle delivery system, to Gu et al., published Nov. 12, 2015.
U.S. Patent Application Publication No. 20100068280 for Sustained release pharmaceutical dosage containing phenylephrine, to Patton et al., published Mar. 18, 2010.
U.S. Patent Application No. 20040162572 for Instrument for surgically cutting tissue and method of use, to Sauer, published Aug. 19, 2004.
U.S. Patent Application No. 20030225460 for Compositions for generating submucosal fluid cushions, to Gostout et al., Dec. 4, 2003.
Wang Z., Liu W., Xu H., Yang X. etc. Preparation and in vitro Studies of Stealth PEGylated PLGA Nanoparticles as Carriers for Arsenic Trioxide. Chin. J. Chem. Eng., 2007; 15: 795-801

Although the foregoing subject matter has been described in some detail by way of illustration and example for purposes of clarity of understanding, it will be understood by those skilled in the art that certain changes and modifications can be practiced within the scope of the appended claims.

That which is claimed:

1. An injectable gel formulation for the sustained release of epinephrine, the formulation comprising epinephrine, or a pharmaceutically acceptable salt thereof, and a degradable polymer or combination of polymers.

2. The injectable gel formulation of claim 1, wherein the degradable polymer or combination of polymers is selected from the group consisting of poly(lactic acid) (PLA), poly(DL-lactide) (PDLA), poly(dl-lactic acid), poly(DL-lactide-co-glycolide) (PLGA), poly(lactic-co-glycolic acid) (PLGA), poly(caprolactone) (PCL), poly(E-caprolactone), poly (ethylene oxide) (PEO), poly(P-dioxanone), poly(hydroxybutyrate), poly(B-malic acid), a poloxamer, poloxamer 407, polycarbophil and Ca++ salt(or equivalent salt), poly(methyl vinyl ether/maleic anhydride), a polyanhydride, a polyphosphazene, a poly(ortho ester), a poly(phosphoester), a polyhydroxyalkanoate (PHA), a polyurethane (PUR), a carbomer, cyclomethicone, chitosan, a triblock PEO-PPO-PEO copolymer of poly(ethylene oxide) (PEO) and poly(propylene oxide) (PPO), oxyethylene oxypropylene polymer (methyl oxirane polymer with oxirane), polyvinylpyrrolidone, alginic acid, Ca alginate and Na salt, agar, xanthan gum, chitosan, chitin, guar gum, a carrageenan, gellan gum, pregelatinized starch, a silk protein polymer, an elastine protein polymer, a silk-elastin protein polymer, collagen, hyaluronic acid, a pseudo-amino acid, albumin, fibrinogen, maltodextrin, tri-block copolymer/poloxamer, and gelatin.

3. The injectable gel formulation of claim 2, comprising a polymer selected from the group consisting of chitosan, a triblock PEO-PPO-PEO copolymer of poly(ethylene oxide) (PEO) and poly(propylene oxide) (PPO), poly(D,L-lactide) (PDLA), poly(D,L-lactide-co-glycolide) (PLGA), oxyethylene oxypropylene polymer (methyl oxirane polymer with oxirane), oxyethylene oxypropylene polymer, poloxamer 407, and polyvinylpyrrolidone.

4. The injectable gel formulation of claim 3, wherein the degradable polymer is methyl oxirane polymer with oxirane or poloxamer 407 alone or in combination with PEO.

5. The injectable gel formulation of claim 2, wherein the degradable polymer comprises from about 10% w/v to about 17% w/v PEO-PPO-PEO triblock copolymer.

6. The injectable gel formulation of claim 2, wherein the degradable polymer comprises from about 0.8% w/v chitosan to about 5% w/v chitosan.

7. The injectable gel formulation of claim 2, wherein the degradable polymer comprises about 2% w/v xanthan gum.

8. The injectable gel formulation of claim 2, wherein the degradable polymer comprises from about 13% w/v to about 17% w/v methyl oxirane polymer with oxirane.

9. The injectable gel formulation of claim 2, wherein the degradable polymer comprises about 10% w/v polyvinylpyrrolidone.

10. The injectable gel formulation of claim 2, wherein the degradable polymer comprises a mixture of about 17% w/v methyl oxirane polymer with oxirane and about 5% w/v chitosan (L) such that the final solution is in a ratio of 17:3 (17 mL of methyl oxirane polymer solution per 3 mL of chitosan(L) solution).

11. The injectable gel formulation of claim 2, wherein the degradable polymer is selected from the group consisting of 1% w/v triblock PEO-PPO-PEO copolymer, 2% w/v triblock PEO-PPO-PEO copolymer, 5% w/v triblock PEO-PPO-PEO copolymer, 10% w/v triblock PEO-PPO-PEO copolymer, 11% w/v triblock PEO-PPO-PEO copolymer, 12% w/v triblock PEO-PPO-PEO copolymer, 13% w/v triblock PEO-PPO-PEO copolymer, 15% w/v triblock PEO-PPO-PEO copolymer, 0.8% w/v chitosan (L), 1% w/v chitosan (L), 1% w/v oxyethylene oxypropylene polymer, 10% w/v oxyethylene oxypropylene polymer, 1% w/v methyl oxirane polymer with oxirane, 10% w/v methyl oxirane polymer with oxirane, 13% w/v methyl oxirane polymer with oxirane, 15% w/v methyl oxirane polymer with oxirane, 1% w/v oxyethylene oxypropylene polymer, 10% w/v oxyethylene oxypropylene polymer, 1% w/v polyvinylpyrrolidone (L), 1% w/v polyvinylpyrrolidone (H), 10% w/v polyvinylpyrrolidone (H), and 15% w/v polyvinylpyrrolidone (H).

12. The injectable gel formulation of claim 1, wherein the gel comprises 6.5% w/v 13% triblock PEO-PPO-PEO copolymers of poly(ethylene oxide) (PEO) and poly(propylene oxide) (PPO), 1.0% w/v poly(ethylene oxide) (PEO), at least one buffer, 0.9% w/v NaCl, at least one dye (0.4 mg), ascorbic acid, and water.

13. The injectable gel formulation of claim 12, further comprising from about 0.001 mg/mL to about 0.1 mg/mL epinephrine, either immediate release or an epinephrine-containing nanoparticle or a combination of both immediate release and an epinephrine-containing nanoparticle.

14. The injectable gel formulation of any one of claims 1-12, wherein the injectable gel comprises a degradable polymer without epinephrine.

15. The injectable gel formulation of claim 1, wherein the epinephrine comprises an epinephrine-containing nanoparticle.

16. A mucoadhesive gel formulation for the sustained release of epinephrine, the formulation comprising epinephrine, a degradable polymer, and a mucoadhesive coating.

17. The mucoadhesive gel formulation of claim 16, wherein the degradable polymer is selected from the group consisting of poly(lactic acid) (PLA), poly(DL-lactide), poly(dl-lactic acid), poly(DL-lactide-co-glycolide), poly(lactic-co-glycolic acid) (PLGA), poly(caprolactone) (PCL), poly(E-caprolactone), poly(P-dioxanone), poly(hydroxybutyrate), poly(B-malic acid), poloxamer, polycarbophil and Ca++ salt, poly(methyl vinyl ether/maleic anhydride), a polyanhydride, a polyphosphazene, a poly(ortho ester), a poly(phosphoester), a polyhydroxyalkanoate (PHA), a polyurethane (PUR), a carbomer, cyclomethicone, alginic acid, Ca alginate and Na salt, agar, xanthan gum, chitosan, chitin, guar gum, a carrageenan, gellan gum, pregelatnaized starch, a silk protein polymer, an elastine protein polymer, a silk-elastin protein polymer, collagen, hyaluronic acid, a pseudo-amino acid, albumin, fibrinogen, maltodextrin, gelatin, polyethylene glycol in combination with acrylated poly-L lactid acid, trilyine amine, albumin, polyethyl amine, glutaraldehyde, polyaldehyde, cyanoacrylate, polyurethane, cyanoacrylate, dextran-urethanemethacrylate, sodium alginate conjugated either with 2-aminoethyl methacrylate, (AEMA), styryl-pyridine, methacrylic anhydride, acrylated poly(glycerol sebacate) (PGS), poly(vinyl acetate) (PVA), PEG, poly(c caprolactone) (PCL), acryloyl chloride (poly(glycerol sebacate acrylate) PGSA), and PEG diacrylate (PEG-DA).

18. The mucoadhesive gel formulation of claim 17, wherein the degradable polymer is poly(lactic-co-glycolic acid (PLGA).

19. The mucoadhesive gel formulation of claim 16, wherein the mucoadhesive coating is selected from the group consisting of chitosan, one or more chitosan salts, and one or more chitosan derivatives.

20. The mucoadhesive gel formulation of claim 19, wherein the mucoadhesive coating comprises chitosan.

21. The mucoadhesive gel formulation of claim 16, further comprising one or more hydrophobic components selected from the group consisting of a synthetic hydrophobic polymer, a naturally-occurring hydrophobic polymer, and combinations thereof.

22. The mucoadhesive gel formulation of claim 21, wherein the synthetic hydrophobic polymer is selected from the group consisting of a polyester, a polyurethane, a polyurea, a polycarbonate, a polyether, a polysulfide, a polysulfonate, a polyimide, a polybenzimidazole, and combinations thereof.

23. The mucoadhesive gel formulation of claim 21, wherein the naturally-occurring hydrophobic polymer is selected from a lipoglycan and a proteoglycan.

24. The mucoadhesive gel formulation of claim 21, wherein the synthetic hydrophobic polymer is selected from the group consisting of a polylactide, polyglycolide, poly(lactide-co-glycolide, poly(e-caprolactone), poly hydroxybutyrate, poly(dioxanone), poly(3-hydroxybutyrate), poly(3-hydroxyval crate), poly(valcrolactone), poly(tartonic acid), poly(malonic acid), poly(anhydrides), poly(orthoesters), polyphosphazenes and acryloyloxy dimethyl-y-butyrolactone (DBA) and other lactone-containing polymers, and combinations thereof.

25. The mucoadhesive gel formulation of claim 21, wherein the hydrophilic polymer is selected from the group consisting of a polyacrylic acid, a polyalcohol, a polyacrylate, a polyurethane, a polyacrylamine, a polyacrylamide, a polyether, and a polypyrollidone.

26. The mucoadhesive gel formulation of claim 21, wherein the hydrophilic polymer comprises one or more monomers selected from the group consisting of acrylate, acrylic acid, methacrylate, methacrylic acid, methyl acrylate, ethyl acrylate, methyl methacrylate, acrylonitrile, 2-chloroethyl vinyl ether, 2-ethylhexyl acrylate, hydroxyethyl methacrylate, butyl acrylate, butyl methacrylate, trimethylolpropane triacrylate, hydroxypropylmethacrylamide, hydroxyethyl acrylate, poly(ethylene glycol) methacrylate, poly(N-isopropylacrylamide) (RNGRAM), poly(vinyl alcohol) (PVA), poly(2-oxazoline), polyethylene glycol, polyvinylpyrollidone polymers, and copolymers thereof.

27. The mucoadhesive gel formulation of claim 16, further comprising one or more boronic acids selected from the group consisting of phenylboronic acid, 2-thienylboronic acid, methylboronic acid, cis-propenylboronic acid, trans-propenylboronic acid, (4-allylaminocarbonyl)benzeneboronic acid, (4-aminosulfonylphenyl)boronic acid, (4-benzyloxy-2-formyl)phenylboronic acid, (4-hydroxy-2-methyl)phenylboronic acid, (4-hydroxy-2-methyl)phenylboronic acid, (4-methanesulfonylaminomethylphenyl)boronic acid, (4-ethanesulfonylaminomethylphenyl)boronic acid, (4-methylaminosulfonylphenyl) boronic acid, (4-methylaminosulfonylphenyl)boronic acid, (4-phenylaminocarbonylphenyl) boronic acid, (4-henylaminocarbonylphenyl)boronic acid, (4-sec-butyl) benzeneboronic acid, (2,6-dimethoxy-4-methylphenyl)boronic acid, (2,6-dimethoxy-4-methylphenyl)boronic acid, (2-methylpropyl)boronic acid, (2-methylpropyl) boronic acid,(3-acetamido-5-carboxy)phenylboronic acid, (3-acetamido-5-carboxy) phenyl boronic acid, (3-acetamidomethylphenyl)boronic acid, (3-acetamidomethylphenyl) boronic acid, (3-allylaminocarbonyl)benzeneboronic acid, (3-cyanomethylphenyl)boronic acid, and derivatives thereof.

28. The mucoadhesive gel formulation of claim 27, wherein the derivative of the one or more boronic esters is selected from the group consisting of allylboronic acid pinacol ester, phenyl boronic acid trimethylene glycol ester, diisopropoxymethylborane, bis(hexyleneglycolato)diboron, t-butyl-N-[4-(4,4,5,5-tetramethyl-1,3,2-dioxaborolan-2-yl)phenyl]carbamate, 2,6-dimethyl-4-(4,4,5,5-tetramethyl-1,3,2-dioxaborolan-2-yl)benzoate, 4-(4,4,5,5-tetramethyl-1,3,2-dioxaborolan-2-yl)aniline, 4-(4,4,5,5-tetramethyl-1,3,2-dioxaborolan-2-yl)benzoic acid, 4-(4,4,5,5-tetramethyl-1,3,2-dioxaborolan-2-yl)phenol, 2-methoxy-4-(4,4,5,5-tetramethyl-1,3,2-dioxaborolan-2-yl)phenol, and combinations thereof.

29. The mucoadhesive gel formulation of claim 16, wherein the degradable polymer has been activated by chemical or UV-A light.

30. The mucoadhesive gel formulation of claim 29, wherein the degradable polymer comprises a mixture of PEO and 3,4-dihydroxyphenyl-L-alanine (DOPA).

31. The mucoadhesive gel formulation of claim 30, wherein the PEO-DOPA mixture further comprises sodium alginate.

32. The mucoadhesive gel formulation of claim 31, wherein the PEO-DOPA mixture comprising sodium alginate has been activated with UV-A light and a photoinitiator.

33. The mucoadhesive gel formulation of claim 32, wherein the photoinitiator is 2-hydroxy-4′-(2-hydroxyethoxy)-2-methylpropiophenone or 2-hydroxy-1-(4-(2-hydroxyethoxy)phenyl)-2-methylpropan-1-one.

34. The mucoadhesive gel formulation of claim 16, wherein the degradable polymer is crosslinked by activation using a divalent or trivalent cation.

35. The mucoadhesive gel formulation of claim 34, wherein the divalent cation is selected from the group consisting of Ca²⁺, Mg²⁺, Ba²⁺, Sr²⁺, Pb²⁺, Cu²⁺, Cd²⁺, Zn²⁺, Ni²⁺, and Co²⁺.

36. The mucoadhesive gel formulation of claim 16, comprising one or more of alginate (Alg), polyethylene oxide (PEO), methacrylic acid, methyl methacrylate (E), hydroxypropylcellulose (HPC), and carboxymethyl cellulose (CMC).

37. The mucoadhesive gel formulation of claim 36, further comprising one or more surfactants.

38. The mucoadhesive gel formulation of claim 37, wherein the one or more surfactants are selected from the group consisting of polyoxyethylene sorbitol ester and sorbitan oleate.

39. The mucoadhesive gel formulation of claim 16, wherein the mucoadhesive gel comprises a formulation selected form the group consisting of 1% w/v sodium alginate, 1.5% w/v sodium alginate, 2.5% w/v sodium alginate, 1% w/v PEO, 2.5% w/v PEO, Alg2.5:PEO1=1:1 with 1% w/v polyoxyethylene sorbitol ester, Alg2.5:PEO1=1:1 with 0.5% w/v polyoxyethylene sorbitol ester, Alg2.5:PEO1=1:1 with 0.5% w/v Sorbitan oleate, Alg2.5: methacrylic acid, and methyl methacrylate=1:1, Alg2.5: methacrylic acid, and methyl methacrylate=1:1, Alg2: methacrylic acid, and methyl methacrylate=3:1, 1.5% w/v sodium alginate, 1% w/v methacrylic acid, and methyl methacrylate, 1.2% w/v sodium alginate, 0.8% w/v methacrylic acid, and methyl methacrylate, 1.5% w/v sodium alginate, 0.1% w/v sodium carboxymethyl cellulose, 1.5% w/v sodium alginate, 0.5% w/v sodium carboxymethyl cellulose, 1.5% w/v sodium alginate, 1% w/v sodium carboxymethyl cellulose, 1.5% w/v sodium alginate, 2.5% w/v PEO, 2% w/v hydroxypropylcellulose, and 4% w/v hydroxypropylcellulose.

40. The mucoadhesive gel formulation of claim 39, comprising 1.5% w/v sodium alginate and 1 M CaCl₂.

41. The mucoadhesive gel formulation of claim 39, comprising 1.2% w/v sodium alginate, 0.8% w/v methacrylic acid, 0.8% w/v methyl methacrylate, and 1 M CaCl₂.

42. The mucoadhesive gel formulation of claim 39, comprising 1.2% w/v sodium alginate, 0.8% w/v methacrylic acid, and 0.8% w/v methyl methacrylate, 0.5% w/v polyoxyethylene sorbitol, a buffer, 0.9% w/v NaCl, a dye, ascorbic acid, 1 M CaCl₂, and water.

43. The mucoadhesive gel formulation of claim 42, further comprising 0.01 mg/mL of epinephrine or an epinephrine-containing nanoparticle.

44. The mucoadhesive gel formulation of claim 39, comprising 1.2% w/v sodium alginate, 0.8% w/v methacrylic acid, 0.8% w/v methyl methacrylate, 0.5% w/v PEO, 0.5% w/v polyoxyethylene sorbitol, a buffer, 0.9% w/v NaCl, a dye, ascorbic acid, and 1 M CaCl₂, and water.

45. The formulation of any one of claims 1-44 further comprising one or more additional therapeutic agents.

46. The formulation of claim 45, wherein the one or more additional therapeutic agents comprises one or more local anesthetics.

47. The formulation of claim 46, wherein the one or more local anesthetics is selected from the group consisting of benzocaine, chloroprocaine, cyclomethycaine, dimethocaine (larocaine), piperocaine, propoxycaine, procaine (novocaine), proparacaine, tetracaine (amethocaine), articaine, bupivacaine, cinchocaine (dibucaine), etidocaine, levobupivacaine, lidocaine (lignocaine), mepivacaine, prilocaine, ropivacaine, trimecaine, saxitoxin, neosaxitoxin, tetrodotoxin, menthol, eugenol, cocaine, spilanthol, and combinations thereof.

48. The formulation of claim 47, wherein the one or more local anesthetics is selected from the group consisting of lidocaine, prilocaine, articaine, and a combination of lidocaine and bupivacaine.

49. The formulation of claim 48, wherein the one or more local anesthetics is lidocaine.

50. The formulation of any one of claims 1-49, further comprising one or more additional components selected from the group consisting of one or more dyes, one or more excipients, one or more buffers, one of more electrolytes, and combinations thereof.

51. The formulation of claim 50, wherein the one or more dyes is selected from the group consisting of indocyanine green, methylene blue, indigo carmine, and combinations thereof.

52. The formulation of claim 50, wherein the one or more excipients is selected from the group consisting of one or more waxes, one or more egg phospholipids, glyceryl monooleate, lecithin, oleic acid, one or more dibutyl sebacate salts, one or more preservatives, glycerin, chlorhexidine, dimethyl sulfoxide, glyceryl behenate, Na stearate, glyceryl palmitostearate, olive oil, sucrose stearate, and combinations thereof.

53. The formulation of claim 50, wherein the one or more buffers is selected from the group consisting of ascorbic acid, maleic acid, tartaric acid, lactic acid, citric acid, acetic acid, sodium bicarbonate, sodium phosphate, and combinations thereof.

54. The formulation of claim 50, wherein the one or more electrolytes is selected from the group consisting of NaCl, KCl, Na₂PO₄, CaPO₄, CaCl₂, and Na Lactate.

55. The mucoadhesive gel formulation of claim 16, wherein the epinephrine comprises free epinephrine or in an epinephrine-containing nanoparticle or a combination of both.

56. A method for preventing or controlling a gastrointestinal bleed, the method comprising administering to a subject in need of treatment thereof a formulation of any one of claims 1-55 or combinations thereof.

57. The method of claim 56, wherein the gastrointestinal bleed is associated with a deep source of gastrointestinal bleeding.

58. The method of claim 57, wherein the deep source of gastrointestinal bleeding is associated with an ulcer.

59. The method of claim 57, wherein the formulation is an injectable formulation of claim 1.

60. The method of claim 56, wherein the gastrointestinal bleed is associated with a superficial source of gastrointestinal bleeding.

61. The method of claim 60, wherein the superficial source of gastrointestinal bleeding is associated with a cancerous lesion.

62. The method of claim 61, wherein the formulation is a mucoadhesive gel formulation of claim 16.

63. The method of claim 62, wherein the mucoadhesive gel binds with sub-mucosa or a tissue defect and shrinks or contracts, thereby approximating the margins of defect and providing a tamponade effect.

64. A method for separating diseased tissue from normal tissue, the method comprising:
(a) injecting a composition of claim 1 under the diseased tissue to form a depot of the composition of claim 1 thereunder, thereby lifting the diseased tissue from the normal tissue; and
(b) dissecting the diseased tissue to separate the diseased tissue from the normal tissue at a dissection site.

65. The method of claim 64, wherein the diseased tissue comprises a polyp.

66. The method of claim 64, comprising an endoscopic procedure selected from the group consisting of endoscopic mucosal resection (EMR), endoscopic sub mucosal dissection (ESD), endoscopic myotomy, third-space endoscopy, endoscopic tunneling, and combinations thereof.

67. The method of claim 64, further comprising administering a mucoadhesive formulation of claim 16 to the dissection site to prevent or control bleeding thereof.

68. An endoscopic injection needle for delivering an injectable solution comprising a mixture of at least two formulations to a tissue treatment site, the endoscopic injection needle comprising:
(a) a connecter comprising a proximal portion and a distal portion:
(i) at least two inlet ports at the proximal portion of the connecter, wherein the at least two inlet ports are in fluid communication with a reservoir;
(ii) an outlet port at the distal portion of the connector, wherein the outlet port is in fluid communication with the reservoir; and
(iii) a plunger movably positionable within the proximal portion of the reservoir, the plunger providing a seal at the proximal portion of the connector to prevent the injectable solution from flowing out of the proximal portion of the connector and wherein the plunger further comprises a plunger advancing member configured to force the injectable solution from the reservoir through the outlet port at the distal portion of the connector;
(b) a static mixing chamber comprising a proximal portion and a distal portion, wherein the proximal portion of the static mixing chamber is in fluid communication with the outlet port at the distal portion of the reservoir, wherein the static mixing chamber is configured to receive the injectable solution from the reservoir; and
(c) a sheath comprising a proximal portion and a distal portion, wherein the proximal portion of the sheath is in fluid communication with the distal portion of the static mixing chamber, and wherein the sheath further comprises a needle enclosed therein, wherein the distal portion of the sheath is movable to expose the needle for insertion into the tissue treatment site.

69. An endoscopic injection needle for delivering an injectable mucoadhesive gel formulation to a tissue treatment site, the endoscopic injection needle comprising:
(a) at least two inlet ports, wherein a first inlet port is in fluid communication with a first chamber for accommodating a mucoadhesive gel and a second inlet port is in fluid communication with a second chamber for accommodating an activator;
(b) at least two outlet channels, wherein a first outlet channel is in fluid communication with the first chamber and a second outlet channel is in fluid communication with the second chamber; and
(c) a first plunger and a second plunger movably positionable within a proximal portion of the first chamber and a proximal portion of the second chamber, the first and second plunger providing a seal at the proximal portion of the first and second chamber to prevent the mucoadhesive gel injectable solution from flowing out of the proximal portion of the first chamber and the activator from flowing out of the proximal portion of the second chamber, wherein the first plunger and the second plunger further comprise a single plunger advancing member configured to force the mucoadhesive gel from the first chamber through the first outlet channel and the activator from the second chamber through the second outlet channel,
wherein the plunger and seal of the second chamber are operationally positioned to form a gap to delay delivery of the activator in relation to delivery of the mucoadhesive gel to the tissue treatment site.

70. A kit comprising a formulation at least one of an injectable gel formulation claim 1, a mucoadhesive gel formulation of claim 16, or combinations thereof.

71. The kit of claim 70, further comprising an endoscopic injection needle of claim 68 or claim 69.

72. A method for delivering one or more therapeutic agents to a targeted site in a gastrointestinal (GI) tract, the method comprising administering a formulation of any one of claims 1-55 with endoscopy to the targeted site.

73. The method of claim 72, wherein the one or more therapeutic agents are selected from the group consisting of one or more corticosteroids, one or more antibiotics, one or more chemotherapeutic agents, one or more tumor necrosis factor inhibitors, one or more angiogenesis inhibitors, one or more kinase inhibitors, one or more immunosuppressive agents, one or more 5-aminosalicylic acid (5-ASA) agents, polytetrafluoroethylene, one or more silicone-based gels, polyacrylamide, polyacrylonitrile, and combinations thereof.

74. The method of claim 72, further comprising treating or preventing one or more diseases, disorders, or conditions selected from the group consisting of one or more strictures in an esophagus or intestine, one or more infected collections around a GI tract, dysmotility or incontinence, inflammatory bowel disease (IBD) and related inflammation, one or more fistulae, and inflammation in liver, pancreas, stomach, intestine, and combinations thereof.

75. A method for sealing a perforation in tissue of a GI tract, the method comprising administering a mucoadhesive gel formulation of claim 16 to the perforated tissue.
